(19) United States
(12) Patent Application Publication
(10) Pub. No.: US 2005/0198747 A1
(43) Pub. Date: Sep. 15, 2005

(54) Use of cationic starch derivatives for dye retention

(76) Inventors: Winfried Emmerling, Tornesch (DE); Gabriele Weser, Kaarst (DE)

Correspondence Address: HENKEL CORPORATION, THE TRIAD, SUITE 200, 2200 RENAISSANCE BLVD., GULPH MILLS, PA, 19406, US

(21) Appl. No.: 11/061,985

(22) Filed: Feb. 18, 2005

Related U.S. Application Data
(60) Provisional application No. 60/548,350, filed on Feb. 27, 2004.

(30) Foreign Application Priority Data
Nov. 13, 2004 (WO) PCT/EP04/12904

(51) Int. Cl. A61K 7/13

(52) U.S. Cl. 8/406

(57) ABSTRACT

Cosmetic compositions methods for their use containing a cationic starch derivative for the treatment of skin and hair. The starch is is a polysaccaride obtained from potatoes, tapioca, cereals or corn.

### CROSS REFERENCE TO RELATED APPLICATIONS

This application claims benefit under 35 U.S.C. §119(e) of United States Provisional Application No. 60,548,350, filed Feb. 27, 2004. This application also claims priority under 35 U.S.C. § 120 of international application PCT/EP2004/012904, filed Nov. 30, 2004.

### BACKGROUND OF THE INVENTION

The invention relates to cosmetic compositions comprising cationic starch derivatives for increasing the dye retention and the washing-out resistance of dyed or bleached keratinic fibers, and the use of these compositions for the cleansing and/or care of the skin and hair and/or for the styling of hair.

The cosmetic treatment of skin and hair is an important part of human body care. Thus, today human hair is treated with hair cosmetic preparations in a variety of ways. These include, for example, the cleansing of the hair using shampoos, care and regeneration using rinses and tonics, and bleaching, dyeing and shaping the hair using colorants, tinting agents, waving agents and styling preparations. Compositions for changing or shading the color of the hair of the head play an outstanding role here. If the bleaching agents, which bring about an oxidative lightening of the hair by breakdown of the natural hair dyes, are disregarded, then in the field of hair dyeing essentially three types of hair colorants are of importance:

For permanent, intense dyeings with corresponding fastness properties, “oxidation dyes” are used. Such colorants customarily contain oxidation dye precursors, “developer components” and “coupler components”. Under the influence of oxidants or of atmospheric oxygen, the developer components form the actual dyes mutually or with coupling to one or more coupler components. The oxidation dyes are distinguished by outstanding, long-lasting dyeing results. For naturally acting dyeings, however, customarily a mixture of a relatively large number of oxidation dye precursors must be employed; in many cases direct dyes are furthermore used for tinting. If the dyes formed in the course of color formation or directly employed have distinctly different fastnesses (e.g. UV stability, perspiration fastness, wash fastness), a recognizable and therefore undesired color shift can occur with time. This phenomenon occurs to an increased extent if the hairstyle contains hair or hair zones with a different degree of damage. An example of this is long hair in which the hair tips exposed long-term to all possible environmental influences as a rule are distinctly more greatly damaged than the relatively freshly rewashed hair zones.

For temporary dyeings, colorants or tinting agents are customarily used which contain “direct sorbers” as the coloring component. These are dye molecules which are absorbed directly on the hair and do not need any oxidative process for the formation of the color. These dyes include, for example, henna, which is already known from antiquity for dyeing the body and hair. These dyeings are as a rule markedly more sensitive than the oxidative dyeings to shampoos, such that a rather undesired shift in tints or even a visible “decolorization” then occurs much more quickly.

Finally, recently a novel dyeing process has found great interest. In this process, precursors of the natural hair dye melanin are applied to the hair; these then form analogs to natural dyes in the course of oxidative processes in the hair. Such a process using 5,6-dihydroxyindoline as the dye precursor was described in EP-B1-530 229. In the case of, in particular multiple, application of compositions containing 5,6-dihydroxyindoline, it is possible to restore the natural hair color to humans having gray hair. The dyeing can be carried out here using atmospheric oxygen as the only oxidant, so that resort does not have to be made to any further oxidant. In the case of persons having originally medium blonde to brown hair, indoline can be employed as the sole dye precursor. For application in the case of persons having an originally red and in particular dark to black hair color, however, satisfactory results can often be obtained only by additional use of further dye components, in particular special oxidation dye precursors.

Not least, due to the great stress on the hair, for example due to dyeing or permanent waving and due to the cleansing of the hair with shampoos and due to environmental stresses, the importance of care products having an action lasting as long as possible increases. Care compositions of this type influence the natural structure and the properties of the hair. Thus, subsequently to such treatments, for example, the wet and dry combability of the hair, the hold and the fullness of the hair can be optimized or the hair can be protected from increased splitting.

It has therefore been customary for a long time to subject the hair to a special aftertreatment. In this treatment, customarily in the form of a rinse, the hair is treated with special active ingredients, for example quaternary ammonium salts or special polymers. As a result of this treatment, depending on the formulation, the combability, the hold and the fullness of the hair are improved and splitting is reduced.

Furthermore, recently “combination preparations” were developed in order to reduce the complexity of the customary multistage processes, in particular in the case of direct application by consumers.

Besides the customary components, for example for cleansing the hair, these preparations additionally contain active ingredients which were formerly reserved for the hair aftertreatment compositions. The consumer is thus spared an application step; at the same time the packaging expenditure is reduced, since one product fewer is used.

The active ingredients available both for separate aftertreatment compositions and for combination preparations in general preferably act on the hair surface. Thus, active ingredients are known which impart luster, hold, fullness, better wet or dry combability to the hair or prevent splitting. Just as important as the external appearance of the hair, however, is the internal structural cohesion of the hair fibers, which can be strongly influenced in particular during oxidative and reductive processes such as dyeing and permanent waving.

The known active ingredients, however, cannot cover all requirements to an adequate extent. There is therefore furthermore a need for active ingredients or active ingredient combinations for cosmetic compositions having good caring properties and good biodegradability. In particular in dye and/or electrolyte-containing formulations, there is a need for additional caring active ingredients which can be incorporated into known formulations without problems.

Starch and starch derivatives are ingredients of cosmetic compositions known per se. Often, however, starch and starch derivatives in particular can only be incorporated very poorly in cosmetic compositions. Thus, for example, the specific adjustment of the viscosity in these compositions is often very difficult, because starch and starch derivatives, as is known, react very sensitively to shear stresses. A further problem is often the elevated temperature necessary for the incorporation of starch and its derivatives. This can lead to undesired adverse effects on the further formulation constituents. Moreover, an additionally necessary process step for heating the cosmetic compositions is associated with increased costs. Lastly, the solubilities of starch and starch derivatives are often inadequate. Surprisingly, it has been shown, however, that when using selected cationic starch derivatives these disadvantages do not exist and additionally these cationic starch derivatives exhibit outstanding properties in cosmetic compositions for the retention of the color fastnesses.

Furthermore, these cationic starch derivatives exhibit outstanding caring properties. Thus, the internal structure of fibers, in particular keratinic fibers, is also significantly restructured. Structure strengthening, that is restructuring within the meaning of the invention, is understood as meaning a reduction of the damage to keratinic fibers resulting due to all sorts of influences. In this context, for example, the restoration of the natural strength plays a significant role. Restructured fibers are distinguished, for example, by an improved luster or by an improved handle or by an easier combability both in the dry and in the wet hair. Additionally, they exhibit optimized strength and elasticity. Successful restructuring can be demonstrated physically, for example, as an increase in melting point in comparison with the damaged fiber. The higher the melting point of the hair, the stronger the structure of the fiber. A detailed description of the method for the determination of the melting range of hair is found in DE 196 173 95 A1.

### DESCRIPTION OF THE INVENTION

The present invention therefore firstly relates to cosmetic preparations comprising a cationic starch derivative for improving the internal and external structure of keratinic fibers and/or for improving the fastness properties of dyed and/or bleached keratinic fibers.

A cationic starch derivative is understood as meaning cationic derivatives of starch. The term starch is understood as meaning polysaccharides which are obtained on a large scale from potatoes, tapioca, cereals or corn. A detailed definition of the term “starch” is found in the textbooks of organic chemistry, for example Beyer/Walter, Lehrbuch der Organischen Chemie [Textbook of organic chemistry], 19th edition, published by Hirzel Verlag Stuttgart. These starch derivatives are modified by the customary processes which are known and described in the literature and finally cationic and starch derivatives are prepared therefrom.

The degree of substitution for the cationic substitution of starch is between 0.01 and 5.0. A degree of substitution of 0.1 to 3.0 is preferred here. Degrees of substitution of 0.1 to 2.0 are very particularly preferred. The molecular weights of the cationic starch derivatives according to the invention are between 100 000 and 50 000 000 daltons. Preferably, the starch derivatives according to the invention have a molecular weight of 100 000 up to 25 000 000 and very particularly preferably from 100 000 up to 20 000 000 daltons.

Suitable cationic starch derivatives can be acquired commercially, for example, under the trade names Starch HP CI 25, Sensomer CI 50, Starch CI 50 A 40, Starch CI 25 MO, Starch CI 25 TA, Starch HP CI 25, Starch HP CI 50 through Ondeo Nalco. Of course, all other cationic starch derivatives which fulfill at least the conditions of the degree of substitution or the molecular weight are also suitable cationic starch derivatives within the meaning of the present invention. Further examples on this matter are found, for example, in “International Cosmetic Ingredient Dictionary and Handbook”, (seventh edition 1997, The Cosmetic, Toiletry, and Fragrance Association 1101 17th Street, N.W., Suite 300, Washington, D.C. 20036-4702).

The use concentration of the modified cationic starch according to the invention in the cosmetic compositions is between 0.05 and 10.0% by weight. Amounts from 0.05 to 7.5% by weight are preferred and amounts from 0.1 to 5.0% by weight are very particularly preferred.

The cationic starch derivatives A used according to the invention significantly improve the important internal and external structural features presented above, the strength and the elasticity of human hair and in particular the fastnesses of dyed and/or bleached keratinic fibers.

It can be preferential according to the invention that the cationic starch derivatives are soluble in the matrix of the cosmetic composition.

Keratinic fibers are understood according to the invention as meaning pelts, wool, feathers and in particular human hair.

In a preferred embodiment of the invention, the action of the cationic starch derivatives (A) according to the invention can be further increased by fatty substances (D). Fatty substances are to be understood as meaning fatty acids, fatty alcohols, natural and synthetic waxes, which can be present both in solid form and liquid in aqueous dispersion, and natural and synthetic cosmetic oil components.

As fatty acids (D1), it is possible to employ linear and/or branched, saturated and/or unsaturated fatty acids having 6-30 carbon atoms. Fatty acids having 10-22 carbon atoms are preferred. Among these, for example, the isostearic acids, such as the commercial products Emersol® 871 and Emersol® 875, and isopalmitic acids such as the commercial product Edenor® IP 95 could be mentioned, and also all other fatty acids marketed under the commercial names Edenor® (Cognis). Further typical examples of such fatty acids are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachic acid, gadoleic acid, behenic acid and erucic acid, and their technical mixtures which are obtained, for example, on the pressure cracking of natural fats and oils, on the oxidation of aldehydes from Roelen's oxo process or the dimerization of unsaturated fatty acids. Customarily, the fatty acid cuts which are obtainable from coconut oil or palm oil are particularly preferred; as a rule, the use of stearic acid is especially preferred.

The amount employed is in this case 0.1-15% by weight, based on the total composition. Preferably, the amount is 0.5-10% by weight, amounts of from 1-5% by weight being very particularly advantageous.

As fatty alcohols (D2), saturated, mono- or poly-unsaturated, branched or unbranched fatty alcohols having C₆-C₃₀, preferably C₁₀-C₂₂ and very particularly preferably C₁₂-C₂₂, carbon atoms can be employed. Within the meaning of the invention, for example, decanol, octanol, octenol, dodecenol, decenol, octadienol, dodecadienol, decadienol, oleyl alcohol, erucyl alcohol, ricinoleyl alcohol, stearyl alcohol, isostearyl alcohol, cetyl alcohol, lauryl alcohol, myristyl alcohol, arachidyl alcohol, caprylyl alcohol, capryl alcohol, linoleyl alcohol, linolenyl alcohol and behenyl alcohol, and their Guerbet alcohols are employable, where this list is to have exemplary and nonlimiting character. The fatty alcohols are derived, however, from preferably natural fatty acids, it customarily being possible to start out from obtainment of the esters of the fatty acids by reduction. According to the invention, those fatty alcohol cuts which are produced by reduction of naturally occurring triglycerides such as beef tallow, palm oil, peanut oil, rapeseed oil, cottonseed oil, soybean oil, sunflower oil and linseed oil or fatty acid esters resulting from their transesterification products with appropriate alcohols, and thus a mixture of different fatty alcohols, are likewise employable. Such substances can be commercially obtained, for example, under the names Stenol®, e.g. Stenol® 1618 or Lanette®, e.g. Lanette® 0 or Lorol®, e.g. Lorol® C8, Lorol® C14, Lorol® C18, Lorol® C8-18, HD-Ocenol®, Crodacol®, e.g. Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 or Isocarb® 24. Of course, wool wax alcohols, such as can be commercially obtained, for example, under the names Corona®, White Swan®, Coronet® or Fluilan®, can also be employed according to the invention. The fatty alcohols are employed in amounts of from 0.1-30% by weight, based on the total preparation, preferably in amounts of from 0.1-20% by weight.

As natural or synthetic waxes (D3), solid paraffins or isoparaffins, carnauba waxes, beeswaxes, candelilla waxes, ozocerite, ceresin, spermaceti, sunflower wax, fruit waxes such as, for example, apple wax or citrus wax, microwaxes consisting of PE- or PP can be employed according to the invention. Waxes of this type are obtainable, for example, through Kahl & Co., Trittau.

The amount employed is 0.1-50% by weight based on the total composition, preferably 0.1-20% by weight and particularly preferably 0.1-15% by weight based on the total composition.

The natural and synthetic cosmetic oily substances (D4), which can increase the action of the starch derivatives (A) according to the invention, include, for example:
vegetable oils. Examples of such oils are sunflower oil, olive oil, soybean oil, rapeseed oil, almond oil, jojoba oil, orange oil, wheatgerm oil, peach kernel oil and the liquid fractions of coconut oil. Also suitable, however, are other triglyceride oils such as the liquid fractions of beef tallow, and synthetic triglyceride oils.
liquid paraffin oils, isoparaffin oils and synthetic hydrocarbons and di-n-alkyl ethers having a total of between 12 and 36 carbon atoms, in particular 12 to 24 carbon atoms, such as, for example, di-n-octyl ether, di-n-decyl ether, di-n-nonyl ether, di-n-undecyl ether, di-n-dodecyl ether, n-hexyl n-octyl ether, n-octyl n-decyl ether, n-decyl n-undecyl ether, n-undecyl n-dodecyl ether and n-hexyl n-undecyl ether, and di-tert-butyl ether, diisopentyl ether, di-3-ethyldecyl ether, tert-butyl n-octyl ether, isopentyl n-octyl ether and 2-methylpentyl n-octyl ether. The compounds 1,3-di(2-ethylhexyl)cyclohexane (Cetiol® S) and di-n-octyl ether (Cetiol® OE) obtainable as commercial products can be preferential.
ester oils. Ester oils are to be understood as meaning the esters of of C₆-C₃₀-fatty acids with C₂-C₃₀-fatty alcohols. The monoesters of fatty acids with alcohols having 2 to 24 carbon atoms are preferred. Examples of fatty acid fractions employed in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachic acid, gadoleic acid, behenic acid and erucic acid, and their technical mixtures which are obtained, for example, in the pressure cracking of natural fats and oils, in the oxidation of aldehydes from Roelen's oxo process or the dimerization of unsaturated fatty acids. Examples of the fatty alcohol fractions in the ester oils are isopropyl alcohol, caproyl alcohol, caprylyl alcohol, 2-ethylhexyl alcohol, capryl alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, eleostearyl alcohol, arachyl alcohol, gadoleyl alcohol, behenyl alcohol, erucyl alcohol and brassidyl alcohol, and their technical mixtures which are obtained, for example, in the high-pressure hydrogenation of technical methyl esters based on fats and oils or aldehydes from Roelen's oxo process, and as a monomer fraction in the dimerization of unsaturated fatty alcohols. According to the invention, isopropyl myristate (Rilanit® BPM), isononanoic acid C₁₆₋₁₈-alkyl ester (Cetiol® SN), 2-ethylhexyl palmitat (Cegesoft® 24) stearic acid 2-ethylhexyl ester (Cetiol® 868), cetyl oleate, glyceryl tricaprylate, coconut fatty alcohol caprate/-caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V) are particularly preferred.
dicarboxylic acid esters such as di-n-butyl adipate, di(2-ethylhexyl) adipate, di(2-ethylhexyl) succinate and diisotridecyl acetate, and diol esters such as ethylene glycol dioleate, ethylene glycol diisotridecanoate, propylene glycol di(2-ethyl hexanoate), propylene glycol diisostearate, propylene glycol dipelargonate, butanediol diisostearate, neopentyl glycol dicaprylate,
symmetrical, unsymmetrical or cyclic esters of carbonic acid with fatty alcohols, described, for example in DE-A 197 56 454, glyceryl carbonate or dicaprylyl carbonate (Cetiol® CC),
trifatty acid esters of saturated and/or unsaturated linear and/or branched fatty acids with glycerol,
fatty acid partial glycerides, that is monoglycerides, diglycerides and their technical mixtures. When using technical products, small amounts of triglycerides associated with preparation can still be contained. The partial glycerides preferably comply with the formula (D4-I), 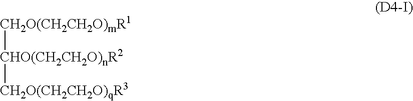
in which R¹, R² and R³, independently of one another, are hydrogen or a linear or branched, saturated and/or unsaturated acyl radical having 6 to 22, preferably 12 to 18, carbon atoms with the proviso that at least one of these groups is an acyl radical and at least one of these groups is hydrogen. The sum (m+n+q) is 0 or numbers from 1 to 100, preferably 0 or 5 to 25. Preferably, R¹ is an acyl radical and R² and R³ are hydrogen and the sum (m+n+q) is 0. Typical examples are mono- and/or diglycerides based on caproic acid, caprylic acid, 2-ethylhexanoic acid, capric acid, lauric acid, isotridecanoic acid, myristic acid, palmitic acid, palmoleic acid, stearic acid, isostearic acid, oleic acid, elaidic acid, petroselinic acid, linoleic acid, linolenic acid, eleostearic acid, arachic acid, gadoleic acid, behenic acid and erucic acid, and their technical mixtures. Preferably, oleic acid monoglycerides are employed.

The amount of the natural and synthetic cosmetic oily substances employed in the compositions used according to the invention is customarily 0.1-30% by weight, based on the total composition, preferably 0.1-20% by weight, and in particular 0.1-15% by weight.

The total amount of oil and fat components in the compositions according to the invention is customarily 0.5-75% by weight, based on the total composition. Amounts of 0.5-35% by weight are preferential according to the invention.

The combination of the cationic starch derivatives (A) according to the invention with surfactants (E) has proven particularly advantageous. In a further preferred embodiment, the compositions used according to the invention contain surfactants. The term surfactants is understood as meaning surface-active substances which form adsorption layers on the surface and interfaces or can aggregate in volume phases to give micellar colloids or lyotropic mesophases. A distinction is made between anionic surfactants consisting of a hydrophobic radical and a negatively charged hydrophilic head group, amphoteric surfactants which carry both a negative and a compensating positive charge, cationic surfactants which in addition to a hydrophobic radical have a positively charged hydrophilic group, and nonionic surfactants which have no charges but strong dipole moments and are strongly hydrated in aqueous solution. More detailed definitions and properties of surfactants can be found in “H.-D. Dörfler, Grenzflächen- und Kolloidchemie [Interface and colloid chemistry], VCH Verlagsgesellschaft mbH. Weinheim, 1994”. The definition of terms reproduced above can be found starting from p. 190 in this publication.

Suitable anionic surfactants (E1) in preparations according to the invention are all anionic surface-active substances suitable for use on the human body. These are characterized by a water-solubilizing, anionic group such as, for example, a carboxylate, sulfate, sulfonate or phosphate group and a lipophilic alkyl group having approximately 8 to 30 carbon atoms. Additionally, glycol or polyglycol ether groups, ester, ether and amide groups, and hydroxyl groups can be present in the molecule. Examples of suitable anionic surfactants are, in each case in the form of the sodium, potassium and ammonium salts, and the mono-, di- and trialkanolammonium salts having 2 to 4 carbon atoms in the alkanol group,
linear and branched fatty acids having 8 to 30 carbon atoms (soaps),
ethercarboxylic acids of the formula R—O—(CH₂—CH₂CO)ₓ—CH₂—COOH, in which R is a linear alkyl group having 8 to 30 carbon atoms and x=0 or 1 to 16,
acylsarcosides having 8 to 24 carbon atoms in the acyl group,
acyltaurides having 8 to 24 carbon atoms in the acyl group,
acylisethionates having 8 to 24 carbon atoms in the acyl group,
sulfosuccinic acid mono- and dialkyl esters having 8 to 24 carbon atoms in the alkyl group and sulfosuccinic acid monoalkyl polyoxyethyl esters having 8 to 24 carbon atoms in the alkyl group and 1 to 6 oxyethyl groups,
linear alkanesulfonates having 8 to 24 carbon atoms,
linear alpha-olefinsulfonates having 8 to 24 carbon atoms,
alpha-sulfo fatty acid methyl esters of fatty acids having 8 to 30 carbon atoms,
alkyl sulfates and alkyl polyglycol ether sulfates of the formula R—O(CH₂—CH₂O)ₓ—OSO₃H, in which R is a preferably linear alkyl group having 8 to 30 carbon atoms and x=0 or 1 to 12,
mixtures of the surface-active hydroxysulfonates according to DE-A-37 25 030,
sulfated hydroxyalkyl polyethylene and/or hydroxy-alkylene propylene glycol ethers according to DE-A-37 23 354,
sulfonates of unsaturated fatty acids having 8 to 24 carbon atoms and 1 to 6 double bonds according to DE-A-39 26 344,
esters of tartaric acid and citric acid with alcohols which are addition products of approximately 2-15 molecules of ethylene oxide and/or propylene oxide to fatty alcohols having 8 to 22 carbon atoms,
alkyl and/or alkenyl ether phosphates of the formula (E1-I), 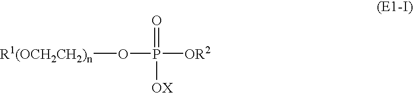
in which R¹ is preferably an aliphatic hydrocarbon radical having 8 to 30 carbon atoms, R² is hydrogen, a radical (CH₂CH₂O)ₙR² or X, n is numbers from 1 to 10 and X is hydrogen, an alkali metal or alkaline earth metal or NR³R⁴R⁵R⁶, with R³ to R⁶ independently of one another being hydrogen or a C₁ to C₄-hydrocarbon radical,
sulfated fatty acid alkylene glycol esters of the formula (E1-II)
   R⁷CO(AlkO)ₙSO₃M (E1-II)
in which R⁷CO— is a linear or branched, aliphatic, saturated and/or unsaturated acyl radical having 6 to 22 carbon atoms, Alk is CH₂CH₂, CHCH₃CH₂ and/or CH₂CHCH₃, n is numbers from 0.5 to 5 and M is a cation, such as are described in DE-A 197 36 906.5,
monoglyceride sulfates and monoglyceride ether sulfates of the formula (E1-III) 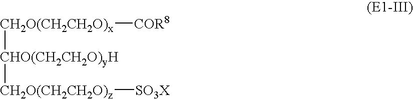
in which R⁸CO is a linear or branched acyl radical having 6 to 22 carbon atoms, x, y and z in total are 0 or numbers from 1 to 30, preferably 2 to 10, and X is an alkali metal or alkaline earth metal. Typical examples of monoglyceride (ether) sulfates suitable within the meaning of the invention are the reaction products of lauric acid monoglyceride, coconut fatty acid monoglyceride, palmitic acid monoglyceride, stearic acid monoglyceride, oleic acid monoglyceride and tallow fatty acid monoglyceride, and their ethylene oxide adducts with sulfur trioxide or chlorosulfonic acid in the form of their sodium salts. Preferably, monoglyceride sulfates of the formula (E1-III) are employed in which R⁸CO is a linear acyl radical having 8 to 18 carbon atoms, such as have been described, for example, in EP-B1 0 561 825, EP-B1 0 561 999, DE-A1 42 04 700 or by A. K. Biswas et al. in J. Am. Oil. Chem. Soc. 37, 171 (1960) and F. U. Ahmed in J. Am. Oil. Chem. Soc. 67, 8 (1990),
amide ethercarboxylic acids such as are described in EP 0 690 044,
condensation products of C₈-C₃₀-fatty alcohols with protein hydrolyzates and/or amino acids and their derivatives, which are known to the person skilled in the art as protein fatty acid condensates, such as, for example, the Lamepon® types, Gluadin® types, Hostapon® KCG or the Amisoft® types.

Preferred anionic surfactants are alkyl sulfates, alkyl polyglycol ether sulfates and ether carboxylic acids having 10 to 18 carbon atoms in the alkyl group and up to 12 glycol ether groups in the molecule, sulfosuccinic acid mono- and dialkyl esters having 8 to 18 carbon atoms in the alkyl group and sulfosuccinic acid monoalkyl polyoxyethyl esters having 8 to 18 carbon atoms in the alkyl group and 1 to 6 oxyethyl groups, monoglyceride sulfates, alkyl and alkenyl ether phosphates, and protein fatty acid condensates.

Zwitterionic surfactants (E2) are designated as those surface-active compounds which carry at least one quaternary ammonium group and at least one —COO⁽⁻⁾ or —SO₃⁽⁻⁾ group in the molecule. Particularly suitable zwitterionic surfactants are the “betaines” such as the N-alkyl-N,N-dimethylammonium glycinates, for example coconut alkyl dimethylammonium glycinate, N-acylaminopropyl-N,N-dimethylammonium glycinates, for example coconut acylaminopropyl dimethylammonium glycinate, and 2-alkyl-3-carboxymethyl-3-hydroxyethyl-imidazoline in each case having 8 to 18 carbon atoms in the alkyl or acyl group, and coconut acylaminoethylhydroxyethylcarboxymethyl glycinate. A preferred zwitterionic surfactant is the fatty acid amide derivative known under the INCI name Cocamidopropyl Betaine.

Ampholytic surfactants (E3) are understood as meaning those surface-active compounds which aside from a C₈-C₂₄-alkyl or -acyl group in the molecule contain at least one free amino group and at least one —COOH or —SO₃H group and are capable of forming internal salts. Examples of suitable ampholytic surfactants are N-alkylglycines, N-alkylpropionic acids, N-alkylaminobutyric acids, N-alkyl-iminodipropionic acids, N-hydroxyethyl-N-alkylamido-propylglycines, N-alkyltaurines, N-alkylsarcosines, 2-alkylaminopropionic acids and alkylaminoacetic acids in each case having approximately 8 to 24 carbon atoms in the alkyl group. Particularly preferred ampholytic surfactants are N-cocoalkylaminopropionate, cocoacylaminoethylaminopropionate and C₁₂-C₁₈-acylsarcosine.

Nonionic surfactants (E4) contain as a hydrophilic group, for example, a polyol group, a polyalkylene glycol ether group or a combination of polyol and polyglycol ether groups. Such compounds are, for example,
addition products of 2 to 50 mol of ethylene oxide and/or 0 to 5 mol of propylene oxide to linear and branched fatty alcohols having 8 to 30 carbon atoms, to fatty acids having 8 to 30 carbon atoms and to alkylphenols having 8 to 15 carbon atoms in the alkyl group,
addition products of 2 to 50 mol of ethylene oxide and/or 0 to 5 mol of propylene oxide which are end group-closed with a methyl or C₂-C₆-alkyl radical to linear and branched fatty alcohols having 8 to 30 carbon atoms, to fatty acids having 8 to 30 carbon atoms and to alkylphenols having 8 to 15 carbon atoms in the alkyl group, such as, for example, the types available under the commercial names Dehydol® LS, Dehydol® LT (Cognis),
C₁₂-C₃₀-fatty acid mono- and diesters of addition products of 1 to 30 mol of ethylene oxide to glycerol,
addition products of 5 to 60 mol of ethylene oxide to castor oil and hardened castor oil,
polyol fatty acid esters, such as, for example, the commercial product Hydagen® HSP (Cognis) or Sovermol types (Cognis),
alkoxylated triglycerides,
alkoxylated fatty acid alkyl esters of the formula (E4-I)
   R¹CO—(OCH₂CHR²)_{w}OR³ (E4-I)
in which R¹CO is a linear or branched, saturated and/or unsaturated acyl radical having 6 to 22 carbon atoms, R² is hydrogen or methyl, R³ is linear or branched alkyl radicals having 1 to 4 carbon atoms and w is numbers from 1 to 20,
amine oxides,
hydroxy mixed ethers, such as are described, for example, in DE-A 19738866,
sorbitan fatty acid esters and addition products of ethylene oxide to sorbitan fatty acid esters such as, for example, the polysorbates,
sugar fatty acid esters and addition products of ethylene oxide to sugar fatty acid esters,
addition products of ethylene oxide to fatty acid alkanolamides and fatty amines,
sugar surfactants of the type consisting of alkyl and alkenyl oligoglycosides according to formula (E4-II),
   R⁴O-[G]ₚ (E4-II)
in which R⁴ is an alkyl or alkenyl radical having 4 to 22 carbon atoms, G is a sugar radical having 5 or 6 carbon atoms and p is numbers from 1 to 10. They can be obtained according to the appropriate processes of preparative organic chemistry. Representative of the extensive literature, reference may be made here to the general study of Biermann et al. in Starch/Stärke 45, 281 (1993), B. Salka in Cosm. Toil. 108, 89 (1993), and J. Kahre et al. in SÖFW-Journal volume 8, 598 (1995).

The alkyl and alkenyl oligoglycosides can be derived from aldoses and ketoses having 5 or 6 carbon atoms, preferably from glucose. The preferred alkyl and/or alkenyl oligoglycosides are thus alkyl and/or alkenyl oligoglucosides. The index number p in the general formula (E4-II) indicates the degree of oligomerization (DP), i.e. the distribution of mono- and oligoglycosides, and is a number between 1 and 10. While p in the individual molecule must always be integral and can here especially assume the values p=1 to 6, the value p for a specific alkyl oligoglycoside is an analytically determined arithmetical variable, which is usually a noninteger. Preferably, alkyl and/or alkenyl oligoglycosides having a mean degree of oligomerization p of 1.1 to 3.0 are employed. From the application technology point of view, those alkyl and/or alkenyl oligoglycosides are preferred whose degree of oligomerization is less than 1.7 and is in particular between 1.2 and 1.4. The alkyl or alkenyl radical R⁴ can be derived from primary alcohols having 4 to 11, preferably 8 to 10, carbon atoms. Typical examples are butanol, caproyl alcohol, caprylyl alcohol, capryl alcohol and undecyl alcohol, and their technical mixtures, such as are obtained, for example, in the hydrogenation of technical fatty acid methyl esters or in the course of hydrogenation of aldehydes from Roelen's oxo process. Preferred alkyl oligoglucosides are those of chain length C₈-C₁₀ (DP=1 to 3), which are obtained as a forerun in the distillative separation of technical C₈-C₁₈-coconut fatty alcohol and can be contaminated with an amount of less than 6% by weight of C₁₂-alcohol, and alkyl oligoglucosides based on technical C_{9/11}-oxo alcohols (DP=1 to 3). The alkyl or alkenyl radical R¹⁵ can furthermore also be derived from primary alcohols having 12 to 22, preferably 12 to 14, carbon atoms. Typical examples are lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol, and their technical mixtures, which can be obtained as described above. Preferred alkyl oligoglucosides are those based on hardened C_{12/14}-coconut alcohol having a DP of 1 to 3.
sugar surfactants of the type consisting of the fatty acid N-alkylpolyhydroxyalkylamides, a nonionic surfactant of the formula (E4-III),
   R⁵CO—NR⁶-[Z] (E4-III)
in which R⁵CO is an aliphatic acyl radical having 6 to 22 carbon atoms, R⁶ is hydrogen, an alkyl or hydroxyalkyl radical having 1 to 4 carbon atoms and [Z] is a linear or branched polyhydroxyalkyl radical having 3 to 12 carbon atoms and 3 to 10 hydroxyl groups. The fatty acid N-alkyl-polyhydroxyalkylamides are known substances, which can customarily be obtained by reductive amination of a reducing sugar using ammonia, an alkylamine or an alkanolamine and subsequent acylation using a fatty acid, a fatty acid alkyl ester or a fatty acid chloride. With respect to the processes for their preparation, reference may be made to the US patents U.S. Pat. No. 1,985,424, U.S. Pat. No. 2,016,962 and U.S. Pat. No. 2,703,798, and the international patent application WO 92/06984. A review on this subject by H. Kelkenberg can be found in Tens. Surf. Det. 25, 8 (1988). Preferably, fatty acid N-alkylpolyhydroxyalkylamides are derived from reducing sugars having 5 or 6 carbon atoms, in particular from glucose. The preferred fatty acid N-alkylpolyhydroxyalkylamides are therefore fatty acid N-alkylglucamides, such as are represented by the formula (E4-IV):
   R⁷CO—NR⁸—CH₂—(CHOH)₄—CH₂OH (E4-IV)
   Preferably, as fatty acid N-alkylpolyhydroxyalkylamides, glucamides of the formula (E4-IV) are employed in which R⁸ is hydrogen or an alkyl group and R⁷CO is the acyl radical of caproic acid, caprylic acid, capric acid, lauric acid, myristic acid, palmitic acid, palmoleic acid, stearic acid, isostearic acid, oleic acid, elaidic acid, petroselinic acid, linoleic acid, linolenic acid, arachic acid, gadoleic acid, behenic acid or erucic acid or their technical mixtures. Particularly preferred are fatty acid N-alkyl-glucamides of the formula (E4-IV), which are obtained by reductive amination of glucose with methylamine and subsequent acylation with lauric acid or C12/14-coconut fatty acid or an appropriate derivative. Furthermore, the polyhydroxyalkylamides can also be derived from maltose and palatinose.

Preferred nonionic surfactants have proven to be the alkylene oxide addition products to saturated linear fatty alcohols and fatty acids in each case having 2 to 30 mol of ethylene oxide per mole of fatty alcohol or fatty acid. Preparations having outstanding properties are likewise obtained if they contain, as nonionic surfactants, fatty acid esters of ethoxylated glycerol.

These compounds are characterized by the following parameters. The alkyl radical R contains 6 to 22 carbon atoms and can be either linear or branched. Primary aliphatic radicals which are linear and methyl-branched in the 2-position are preferred. Such alkyl radicals are, for example, 1-octyl, 1-decyl, 1-lauryl, 1-myristyl, 1-cetyl and 1-stearyl. 1-Octyl, 1-decyl, 1-lauryl, 1-myristyl are particularly preferred. When using “oxo alcohols” as starting substances, compounds having an odd number of carbon atoms in the alkyl chain predominate.

Furthermore, the sugar surfactants are very particularly preferred nonionic surfactants. These can be present in the compositions used according to the invention preferably in amounts of 0.1-20% by weight, based on the total composition. Amounts of 0.5-15% by weight are preferred, and amounts of 0.5-7.5% by weight are very particularly preferred.

The compounds having acyl groups employed as a surfactant can in each case be homogeneous substances. It is, however, as a rule preferred in the preparation of these substances to start out from native vegetable or animal raw materials, such that substance mixtures having different alkyl chain lengths in each case dependent on the raw material are obtained.

In the case of the surfactants which are addition products of ethylene oxide and/or propylene oxide to fatty alcohols or derivatives of these addition products, either products having a “normal” homolog distribution or those having a concentrated homolog distribution can be used. “Normal” homolog distribution is understood here as meaning mixtures of homologs which are obtained in the reaction of fatty alcohol and alkylene oxide using alkali metals, alkali metal hydroxides or alkali metal alkoxides as catalysts. Concentrated homolog distributions are, in contrast, obtained if, for example hydrotalcites, alkaline earth metal salts of ethercarboxylic acids, alkaline earth metal oxides, hydroxides or alkoxides are used as catalysts. The use of products having a concentrated homolog distribution can be preferential.

The surfactants (E) are employed in amounts of from 0.1-45% by weight, preferably 0.5-30% by weight and very particularly preferably of 0.5-25% by weight, based on the total composition used according to the invention.

Likewise employable according to the invention are cationic surfactants (E5) of the type consisting of the quaternary ammonium compounds, the ester quats and the amidoamines. Preferred quaternary ammonium compounds are ammonium halides, in particular chlorides and bromides, such as alkyltrimethylammonium chlorides, dialkyldimethylammonium chlorides and trialkylmethylammonium chlorides, e.g. cetyltrimethylammonium chloride, stearyltrimethylammonium chloride, distearyldimethylammonium chloride, lauryldimethylammonium chloride, lauryldimethylbenzylammonium chloride and tricetylmethylammonium chloride, and the imidazolium compounds known under the INCI names Quaternium-27 and Quaternium-83. The long alkyl chains of the abovementioned surfactants preferably have 10 to 18 carbon atoms.

Ester quats are known substances which contain both at least one ester function and at least one quaternary ammonium group as a structural element. Preferred ester quats are quaternized ester salts of fatty acids with triethanolamine, quaternized ester salts of fatty acids with diethanolalkylamines and quaternized ester salts of fatty acids with 1,2-dihydroxypropyldialkylamines. Such products are marketed, for example, under the trademarks Stepantex®, Dehyquart® and Armocare®. The products Armocare® VGH-70, an N,N-bis(2-palmitoyloxy-ethyl)dimethylammonium chloride, and Dehyquart® F-75, Dehyquart® C-4046, Dehyquart® L80 and Dehyquart® AU-35 are examples of such ester quats.

The alkylamidoamines are customarily prepared by amidation of natural or synthetic fatty acids and fatty acid cuts with dialkylaminoamines. A particularly suitable compound according to the invention from this substance group is the stearamidopropyldimethylamine commercially obtainable under the name Tegoamid® S 18.

The cationic surfactants (E5) are present in the compositions used according to the invention preferably in amounts of from 0.05 to 10% by weight, based on the total composition. Amounts of 0.1 to 5% by weight are particularly preferred.

Anionic, nonionic, zwitterionic and/or amphoteric surfactants and their mixtures can be preferential according to the invention.

In a further preferred embodiment, the action of the cationic starch derivatives (A) according to the invention can be increased by emulsifiers (F). Emulsifiers bring about the formation of water- or oil-stable adsorption layers on the phase interface, which protect the dispersed droplets against coalescence and thus stabilize the emulsion. Emulsifiers are therefore constructed, like surfactants, from a hydrophobic and a hydrophilic moiety. Hydrophilic emulsifiers preferably form O/W emulsions and hydrophobic emulsifiers preferably form W/O emulsions. An emulsion is to be understood as meaning a droplet-like dispersion of a liquid in another liquid with the expenditure of energy for the creation of stabilizing phase interfaces by means of surfactants. The selection of these emulsifying surfactants or emulsifiers depends here on the substances to be dispersed and the respective external phase, and the finely divided nature of the emulsion. More extensive definitions and properties of emulsifiers can be found in “H.-D. Dörfler, Grenzflächen- and Kolloidchemie, VCH Verlagsgesellschaft mbH, Weinheim, 1994”. Emulsifiers utilizable according to the invention are, for example,
addition products of 4 to 30 mol of ethylene oxide and/or 0 to 5 mol of propylene oxide to linear fatty alcohols having 8 to 22 carbon atoms, to fatty acids having 12 to 22 carbon atoms and to alkylphenols having 8 to 15 carbon atoms in the alkyl group,
C₁₂-C₂₂-fatty acid mono- and diesters of addition products of 1 to 30 mol of ethylene oxide to polyols having 3 to 6 carbon atoms, in particular to glycerol,
ethylene oxide and polyglycerol addition products to methyl glucoside fatty acid esters, fatty acid alkanolamides and fatty acid glucamides,
C₈-C₂₂-alkylmono- and oligoglycosides and their ethoxylated analogs, degrees of oligomerization of 1.1 to 5, in particular 1.2 to 2.0, and glucose as the sugar component being preferred,
mixtures of alkyl (oligo)glucosides and fatty alcohols, for example the commercially obtainable product Montanov®68,
addition products of 5 to 60 mol of ethylene oxide to castor oil and hardened castor oil, partial esters of polyols having 3-6 carbon atoms with saturated fatty acids having 8 to 22 carbon atoms,
sterols. Sterols is understood as meaning a group of steroids which carry a hydroxyl group on the carbon atom 3 of the steroid structure and are isolated either from animal tissue (zoosterols) or from vegetable fats (phytosterols). Examples of zoosterols are cholesterol and lanosterol. Examples of suitable phytosterols are ergosterol, stigmasterol and sitosterol. Sterols, the “mycosterols”, are also isolated from fungi and yeasts.
phospholipids. These are especially understood as meaning the glucose phospholipids, which are obtained, for example, as lecithins or phosphatidylcholines from, for example, egg yolks or plant seeds (e.g. soybeans).
fatty acid esters of sugars and sugar alcohols, such as sorbitol,
polyglycerols and polyglycerol derivatives such as, for example, polyglycerol poly-12-hydroxystearate (commercial product Dehymuls® PGPH),
linear and branched fatty acids having 8 to 30 carbon atoms and their Na, K, ammonium, Ca, Mg and Zn salts.

The compositions according to the invention contain the emulsifiers preferably in amounts of from 0.1-25% by weight, in particular 0.5-15% by weight, based on the total composition.

Preferably, the compositions according to the invention can contain at least one nonionic emulsifier having an HLB of 8 to 18, according to the definitions listed in Römpp-Lexikon Chemie [Römpp's encyclopedia of chemistry] (ed. J. Falbe, M. Regitz), 10th edition, Georg Thieme Verlag Stuttgart, N.Y., (1997), page 1764. Nonionic emulsifiers having an HLB of 10-15 can be particularly preferential according to the invention.

It has furthermore been shown to be advantageous that polymers (G) can assist the action of the cationic starch derivatives (A) according to the invention. In a preferred embodiment, polymers are therefore added to the compositions used according to the invention, with cationic, anionic, amphoteric and nonionic polymers having proven effective.

Cationic polymers (G1) are to be understood as meaning polymers which contain a group in the main chain and/or side chain which can be “temporarily” or “permanently” cationic. Those polymers which independently of the pH of the composition have a cationic group are designated according to the invention as “permanently cationic”. These are as a rule polymers which contain a quaternary nitrogen atom, for example in the form of an ammonium group. Preferred cationic groups are quaternary ammonium groups. In particular, those polymers in which the quaternary ammonium group is bonded via a C₁₋₄-hydrocarbon group to a main polymer chain constructed from acrylic acid, methacrylic acid or their derivatives have proven particularly suitable.

Homopolymers of the general formula (G1-I), 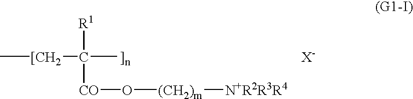
in which R¹=—H or —CH₃, R², R³ and R⁴, independently of one another, are selected from C₁₋₄-alkyl, -alkenyl or -hydroxyalkyl groups, m=1, 2, 3 or 4, n is a natural number and X⁻ is a physiologically tolerable organic or inorganic anion, and copolymers consisting essentially of the monomer units and nonionic monomer units shown in formula (G1-I) are particularly preferred cationic polymers. In the context of these polymers, those are preferred according to the invention for which at least one of the following conditions applies:
R¹ is a methyl group
R², R³ and R⁴ are methyl groups
m has the value 2.

Suitable physiologically tolerable counterions X⁻ are, for example, halide ions, sulfate ions, phosphate ions, methosulfate ions, and organic ions such as lactate, citrate, tartrate and acetate ions. Halide ions are preferred, in particular chloride.

A particularly suitable homopolymer is the, if desired crosslinked, poly(methacryloyloxyethyltrimethylammonium chloride) having the INCI name Polyquaternium-37. The crosslinking can be carried out if desired with the aid of polyolefinically unsaturated compounds, for example divinylbenzene, tetraallyloxyethane, methylene bisacrylamide, diallyl ether, polyallyl polyglyceryl ether, or allyl ethers of sugars or sugar derivatives such as erythritol, pentaerythritol, arabitol, mannitol, sorbitol, sucrose or glucose. Methylene bisacrylamide is a preferred crosslinking agent.

The homopolymer is preferably employed in the form of a nonaqueous polymer dispersion, which should have a polymer content of not below 30% by weight. Such polymer dispersions are commercially obtainable under the names Salcare® SC 95 (about 50% polymer content, further components: mineral oil (INCI name: Mineral Oil) and tridecyl polyoxypropylene-polyoxyethylene ether (INCI name: PPG-1-Trideceth-6)) and Salcare® SC 96 (about 50% polymer content, further components: mixture of diesters of propylene glycol with a mixture of caprylic and capric acid (INCI name: Propylene Glycol Dicaprylate/Dicaprate) and tridecyl polyoxypropylene-polyoxyethylene ether (INCI name: PPG-1-Trideceth-6)).

Copolymers containing monomer units according to formula (G1-I) preferably contain as nonionic monomer units acrylamide, methacrylamide, acrylic acid C₁₋₄-alkyl esters and methacrylic acid C₁₋₄-alkyl esters. Among these nonionic monomers, acrylamide is particularly preferred. These copolymers can also be crosslinked, as described above in the case of the homopolymers. A preferred copolymer according to the invention is the crosslinked acrylamide-methacryloyloxyethyltrimethylammonium chloride copolymer. Such copolymers, in which the monomers are present in a weight ratio of approximately 20:80, are obtainable commercially as an approximately 50% strength nonaqueous polymer dispersion under the name Salcare® SC 92.

Further preferred cationic polymers are, for example,
quaternized cellulose derivatives, such as are commercially obtainable under the names Celquat® and Polymer JR®. The compounds Celquat® H 100, Celquat® L 200 and Polymer JR®400 are preferred quaternized cellulose derivatives,
cationic alkyl polyglycosides according to DE-C 44 13 686,
cationized honey, for example the commercial product Honeyquat® 50,
cationic guar derivatives, such as, in particular, the products marketed under the commercial names Cosmedia®Guar and Jaguar®,
polysiloxanes having quaternary groups, such as, for example, the commercially obtainable products Q2-7224 (manufacturer: Dow Corning; a stabilized trimethylsilylamodimethicone), Dow Corning® 929 Emulsion (comprising a hydroxylamino-modified silicone, which is also designated as amodimethicone), SM-2059 (manufacturer: General Electric), SLM-55067 (manufacturer: Wacker) and Abil®-Quat 3270 and 3272 (manufacturer: Th. Goldschmidt), diquaternary polydimethylsiloxane (Quaternium-80),
polymeric dimethyldiallylammonium salts and their copolymers with esters and amides of acrylic acid and methacrylic acid. The products commercially obtainable under the names Merquat® 100 (poly(dimethyldiallylammonium chloride)) and Merquat®550 (dimethyldiallylammonium chloride-acrylamide copolymer) are examples of such cationic polymers,
copolymers of vinylpyrrolidone with quaternized derivatives of dialkylaminoalkyl acrylate and methacrylate, such as, for example vinylpyrrolidone-dimethylaminoethyl methacrylate copolymers quaternized with diethyl sulfate. Such compounds are commercially obtainable under the names Gafquat®734 and Gafquat®755,
vinylpyrrolidone-vinylimidazolium methochloride copolymers, such as are supplied under the names Luviquat® FC 370, PC 550, FC 905 and HM 552,
quaternized polyvinyl alcohol,
and the polymers having quaternary nitrogen atoms in the main polymer chain known under the names Polyquaternium 2, Polyquaternium 17, Polyquaternium 18 and Polyquaternium 27.

The polymers known under the names Polyquaternium-24 (commercial product, for example Quatrisoft® LM 200), can likewise be employed as cationic polymers. The copolymers of vinylpyrrolidone, such as are obtainable as the commercial products Copolymer 845 (manufacturer: ISP), Gaffix® VC 713 (manufacturer ISP), Gafquat®ASCP 1011, Gafquat®HS 110, Luviquat®8155 and Luviquat® MS 370 are likewise utilizable according to the invention.

Further cationic polymers according to the invention are the “temporarily cationic” polymers. These polymers customarily contain an amino group, which is present as a quaternary ammonium group and thus cationic at certain pHs. For example, chitosan and its derivatives are preferred, such as are commercially freely available, for example, under the trade names Hydagen® CMF, Hydagen® HCMF, Kytamer® PC and Chitolam® NB/101.

Cationic polymers preferred according to the invention are cationic cellulose derivatives and chitosan and its derivatives, in particular the commercial products Polymer® JR 400, Hydagen® HCMF and Kytamer® PC, cationic guar derivatives, cationic honey derivatives, in particular the commercial product Honeyquat® 50, cationic alkyl polyglycosides according to DE-C 44 13 686 and polymers of the type Polyquaternium-37.

Furthermore, cationized protein hydrolyzates are to be counted among the cationic polymers, where the underlying protein hydrolyzates can be derived from animals, for example from collagen, milk or keratin, from plants, for example from wheat, corn, rice, potatoes, soybeans or almonds, from marine life forms, for example from fish collagen or algae, or biotechnologically obtained protein hydrolyzates. The protein hydrolyzates on which the cationic derivatives according to the invention are based can be obtained from the corresponding proteins by a chemical, in particular alkaline or acidic hydrolysis, by an enzymatic hydrolysis and/or a combination of both types of hydrolysis. The hydrolysis of proteins as a rule affords a protein hydrolyzate having a molecular weight distribution from approximately 100 daltons to up to several thousand daltons. Those cationic protein hydrolyzates are preferred whose underlying protein component has a molecular weight of 100 up zu 25 000 daltons, preferably 250 to 5000 daltons. Furthermore, cationic protein hydrolyzates are to be understood as meaning quaternized amino acids and their mixtures. The quaternization of the protein hydrolyzates or of the amino acids is often carried out by means of quaternary ammonium salts such as, for example, N,N-dimethyl-N-(n-alkyl)-N-(2-hydroxy-3-chloro-n-propyl)ammonium halides. Furthermore, the cationic protein hydrolyzates can also be derivatized even further. As typical examples of the cationic protein hydrolyzates and derivatives according to the invention, the products mentioned under the INCI names in the “International Cosmetic Ingredient Dictionary and Handbook”, (seventh edition 1997, The Cosmetic, Toiletry, and Fragrance Association 1101 17th Street, N.W., Suite 300, Washington, D.C. 20036-4702) and commercially obtainable may be mentioned: Codimonium Hydroxypropyl Hydrolyzed Collagen, Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Soy Protein, Cocodimonium Hydroxypropyl Hydrolyzed Wheat Protein, Hydroxypropyl Arginine Lauryl/Myristyl Ether HCl, Hydroxy-Propyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed Keratin, Hydroxypropyltrimonium Hydrolyzed Rice Bran Protein, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxy-Silicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Soy Protein, Quaternium-79 Hydrolyzed Wheat Protein.

The cationic protein hydrolyzates and derivatives based on plants are very particularly preferred.

The anionic polymers (G2) which can assist the action of the cationic starch derivatives (A) according to the invention are anionic polymers which contain carboxylate and/or sulfonic acid groups. Examples of anionic monomers from which polymers of this type can consist are acrylic acid, methacrylic acid, crotonic acid, maleic anhydride and 2-acrylamido-2-methylpropanesulfonic acid. Here, the acidic groups can be present completely or partly as the sodium, potassium, ammonium, mono- or triethanolammonium salt. Preferred monomers are 2-acrylamido-2-methylpropanesulfonic acid and acrylic acid.

Very particularly effective anionic polymers have proven to be those which, as the sole or comonomer, contain 2-acrylamido-2-methylpropanesulfonic acid, where the sulfonic acid group can be present completely or partly as the sodium, potassium, ammonium, mono- or triethanolammonium salt.

Particularly preferred is the homopolymer of 2-acrylamido-2-methylpropanesulfonic acid, which is commercially obtainable, for example, under the name Rheothik® 11-80.

Within this embodiment, it can be preferential to employ copolymers of at least one anionic monomer and at least one nonionic monomer. With respect to the anionic monomers, reference is made to the abovementioned substances. Preferred nonionic monomers are acrylamide, methacrylamide, acrylic acid esters, methacrylic acid esters, vinylpyrrolidone, vinyl ethers and vinyl esters.

Preferred anionic copolymers are acrylic acid-acrylamide copolymers and in particular polyacrylamide copolymers with monomers containing sulfonic acid groups. A particularly preferred anionic copolymer consists of 70 to 55 mol % of acrylamide and 30 to 45 mol % of 2-acrylamido-2-methylpropanesulfonic acid, where the sulfonic acid group can be present completely or partly as the sodium, potassium, ammonium, mono- or triethanolammonium salt. This copolymer can also be present crosslinked, where the crosslinking agents preferably employed are polyolefinically unsaturated compounds such as tetraallyloxyethane, allylsucrose, allylpentaerythritol and methylenebisacrylamide. Such a polymer is contained in the commercial product Sepigel®305 from SEPPIC. The use of this compound, which in addition to the polymer component contains a hydrocarbon mixture (C₁₃-C₁₄-isoparaffin) and a nonionic emulsifier (Laureth-7), has proven particularly advantageous in the context of the teaching according to the invention.

The sodium acryloyldimethyltaurate copolymers marketed under the name Simulgel®600 as a compound with isohexadecane and polysorbate-80 have also proven to be particularly effective according to the invention.

Likewise preferred anionic homopolymers are uncrosslinked and crosslinked polyacrylic acids. Allyl ethers of pentaerythritol, of sucrose and of propylene can be the preferred crosslinking agents here. Such compounds are commercially obtainable, for example, under the trademark Carbopol®.

Copolymers of maleic anhydride and methyl vinyl ether, in particular those having crosslinkages, are likewise dye-retaining polymers. A maleic acid-methyl vinyl ether copolymer crosslinked with 1,9-decadiene is commercially obtainable under the name Stabileze® QM.

Furthermore, amphoteric polymers (G3) can be used as polymers for increasing the action of the cationic starch derivatives (A) according to the invention. Under the term amphoteric polymers are classified both those polymers which contain both free amino groups and free —COOH or SO₃H groups in the molecule and are capable of forming internal salts, and zwitterionic polymers which contain quaternary ammonium groups and —COO⁻ or —SO₃⁻ groups in the molecule, and those polymers which contain —COOH or SO₃H groups and quaternary ammonium groups.

An example of an amphopolymer which is employable according to the invention is the acrylic resin obtainable under the name Amphomer®, which is a copolymer of tert-butylaminoethyl methacrylate, N-(1,1,3,3-tetramethylbutyl)acrylamide, and two or more monomers from the group consisting of acrylic acid, methacrylic acid and their simple esters.

Further amphoteric polymers which are employable according to the invention are the compounds mentioned in the British laid-open specification 2 104 091, the European laid-open specification 47 714, the European laid-open specification 217 274, the European laid-open specification 283 817 and the German laid-open specification 28 17 369.

Amphoteric polymers preferably employed are those polymers which are composed essentially of
(a) monomers having quaternary ammonium groups of the general formula (G3-I),
   R¹CH═CR²—CO-Z-(CₙH₂ₙ)—N⁽⁺⁾R³N⁴R⁵A⁽⁻⁾ (G3-I)
   in which R¹ and R independently of one another are hydrogen or a methyl group and R³, R⁴ and R⁵ independently are alkyl groups having 1 to 4 carbon atoms, Z is an NH group or an oxygen atom, n is an integer from 2 to 5 and A⁽⁻⁾ is the anion of an organic or inorganic acid, and
(b) monomeric carboxylic acids of the general formula (G3-II),
   R⁶CH═CR⁷—COOH (G3-II)
   in which R⁶ and R⁷ independently of one another are hydrogen or methyl groups.

These compounds can be employed according to the invention both directly and in salt form, which is obtained by neutralization of the polymers, for example with an alkali metal hydroxide. With respect to the details of the preparation of these polymers, reference is expressly made to the contents of the German laid-open specification 39 29 973. Very particularly preferred polymers are those in which monomers of the type (a) are employed in which R³, R⁴ and R⁵ are methyl groups, Z is an NH group and A⁽⁻⁾ is a halide, methoxysulfate or ethoxysulfate ion; acrylamido-propyltrimethylammonium chloride is a particularly preferred monomer (a). The monomer (b) used for the polymers mentioned is preferably acrylic acid.

In a further embodiment, the compositions according to the invention can contain nonionic polymers (G4).

Suitable nonionic polymers are, for example:
vinylpyrrolidone/vinyl ester copolymers, such as are marketed, for example, under the trademark Luviskol® (BASF). Luviskol® VA 64 and Luviskol® VA 73, in each case vinylpyrrolidone/vinyl acetate copolymers, are likewise preferred nonionic polymers.
cellulose ethers, such as hydroxypropylcellulose, hydroxyethylcellulose and methylhydroxypropylcellulose, such as are marketed, for example, under the trademarks Culminal® and Benecel® (AQUALON)
shellac
polyvinylpyrrolidones, such as are marketed, for example, under the name Luviskol® (BASF).
siloxanes. These siloxanes can be either water-soluble or water-insoluble. Both volatile and nonvolatile siloxanes are suitable, nonvolatile siloxanes being understood as meaning those compounds whose boiling point at normal pressure is above 200° C. Preferred siloxanes are polydialkylsiloxanes, such as, for example, polydimethylsiloxane, polyalkylarylsiloxanes, such as, for example, polyphenylmethylsiloxane, ethoxylated polydialkylsiloxanes, and polydialkyl-siloxanes which contain amine and/or hydroxy groups.
glycosidically substituted silicones according to EP 0612759 B1.

According to the invention, it is also possible that the preparations used contain a number of, in particular two, different polymers of identical charge and/or in each case an ionic and an amphoteric and/or nonionic polymer.

The polymers (G) are present in the compositions used according to the invention preferably in amounts of 0.05 to 10% by weight, based on the total composition. Amounts of from 0.1 to 5, in particular from 0.1 to 3% by weight, are particularly preferred.

Furthermore, in a preferred embodiment of the invention the action of the cationic starch derivatives (A) can be increased by UV filters (I). The UV filters to be used according to the invention are subject to no general restrictions with respect to their structure and their physical properties. Rather, all UV filters employable in the cosmetic field whose absorption maximum is in the UVA (315-400 nm), in the UVB (280-315 nm) or in the UVC (<280 nm) range are suitable. UV filters having an absorption maximum in the UVB range, in particular in the range from approximately 280 to approximately 300 nm, are particularly preferred.

The UV filters used according to the invention can be selected, for example, from substituted benzophenones, p-aminobenzoic acid esters, diphenylacrylic acid esters, cinnamic acid esters, salicylic acid esters, benzimidazoles and o-aminobenzoic acid esters.

Examples of UV filters utilizable according to the invention are 4-aminobenzoic acid, N,N,N-trimethyl-4-(2-oxoborn-3-ylidenemethyl)aniline methylsulfate, 3,3,5-trimethylcyclohexyl salicylate (Homosalate), 2-hydroxy-4-methoxybenzophenone (Benzophenone-3; Uvinul®M 40, Uvasorb®MET, Neo Heliopan®BB, Eusolex®4360), 2-phenylbenzimidazole-5-sulfonic acid and its potassium, sodium and triethanolamine salts (phenyl-benzimidazolesulfonic acid; Parsol®HS; Neo Heliopan®Hydro), 3,3′-(1,4-phenyl-enedimethylene)bis(7,7-dimethyl-2-oxobicyclo-[2.2.1]hept-1-ylmethanesulfonic acid) and its salts, 1-(4-tert-butylphenyl)-3-(4-methoxyphenyl)-propane-1,3-dione (Butyl methoxydibenzoylmethane; Parsol®1789, Eusolex®9020), α-(2-oxoborn-3-ylidene)-toluene-4-sulfonic acid and its salts, ethoxylated ethyl 4-aminobenzoate (PEG-25 PABA; Uvinul®P 25), 2-ethylhexyl 4-dimethylaminobenzoate (Octyl Dimethyl PABA; Uvasorb®DMO, Escalol®507, Eusolex® 6007), 2-ethylhexyl salicylate (Octyl Salicylate; Escalol®587, Neo Heliopan®OS, Uvinul®O18), isopentyl 4-methoxycinnamate (Isoamyl p-Methoxycinnamate; Neo Heliopan®E 1000), 2-ethylhexyl 4-methoxycinnamate (Octyl Methoxycinnamate; Parsol®MCX, Escalol®557, Neo Heliopan®AV), 2-hydroxy-4-methoxybenzophenone-5-sulfonic acid and its sodium salt (Benzophenone-4; Uvinul®MS 40; Uvasorb®S 5), 3-(4′-methylbenzylidene)-D,L-camphor (4-methylbenzylidene camphor; Parsol®5000, Eusolex®6300), 3-benzylidenecamphor (3-Benzylidene camphor), 4-isopropylbenzyl salicylate, 2,4,6-trianilino-(p-carbo-2′-ethylhexyl-1′-oxy)-1,3,5-triazine, 3-imidazol-4-ylacrylic acid and its ethyl esters, polymers of N-{(2 and 4)-[2-oxoborn-3-ylidenemethyl]benzyl}acrylamide, 2,4-dihydroxybenzophenone (Benzophenone-1; Uvasorb®20H, Uvinul®400), 1,1′-diphenylacrylonitrilic acid ethylhexyl ester (Octocrylene; Eusolex®OCR, Neo Heliopan®Type 303, Uvinul®N 539 SG), o-aminobenzoic acid menthyl ester (Menthyl Anthranilate; Neo Heliopan®MA), 2,2′,4,4′-tetrahydroxybenzophenone (Benzophenone-2; Uvinul®D-50), 2,2′-dihydroxy-4,4′-dimethoxybenzophenone (Benzophenone-6), 2,2′-dihydroxy-4,4′-dimethoxybenzo-phenone-5-sodium sulfonate and 2-cyano-3,3-diphenylacrylic acid 2′-ethylhexyl ester. 4-Aminobenzoic acid, N,N,N-trimethyl-4-(2-oxoborn-3-ylidenemethyl)aniline methylsulfate, 3,3,5-trimethylcyclohexyl salicylate, 2-hydroxy-4-methoxybenzophenone, 2-phenylbenzimidazole-5-sulfonic acid and its potassium, sodium and triethanolamine salts, 3,3′-(1,4-phenylenedimethylene)-bis(7,7-dimethyl-2-oxobicyclo[2.2.1]hept-1-ylmethanesulfonic acid) and its salts, 1-(4-tert-butylphenyl)-3-(4-methoxyphenyl)propane-1,3-dione, α-(2-oxoborn-3-ylidene)toluene-4-sulfonic acid and its salts, ethoxylated 4-aminobenzoic acid ethyl ester, 4-dimethylaminobenzoic acid 2-ethylhexyl ester, salicylic acid 2-ethylhexyl ester, 4-methoxycinnamic acid isopentyl ester, 4-methoxycinnamic acid 2-ethylhexyl ester, 2-hydroxy-4-methoxybenzophenone-5-sulfonic acid and its sodium salt, 3-(4′-methylbenzylidene)-D,L-camphor, 3-benzylidenecamphor, 4-isopropylbenzyl salicylate, 2,4,6-trianilino-(p-carbo-2′-ethylhexyl-1′-oxy)-1,3,5-triazine, 3-imidazol-4-ylacrylic acid and its ethyl ester, polymers of N-{(2 and 4)-[2-oxoborn-3-ylidenemethyl]benzyl}acrylamide are preferred. According to the invention, 2-hydroxy-4-methoxybenzo-phenone, 2-phenylbenzimidazole-5-sulfonic acid and its potassium, sodium and triethanolamine salts, 1-(4-tert-butylphenyl)-3-(4-methoxyphenyl)propane-1,3-dione, 4-methoxycinnamic acid 2-ethylhexyl ester and 3-(4′-methylbenzylidene)-D,L-camphor are very particularly preferred.

Those UV filters whose molar extinction coefficient at the absorption maximum is above 15 000, in particular above 20 000, are preferred.

It was furthermore found that with structurally similar UV filters in many cases the water-insoluble compound in the context of the teaching according to the invention has the higher action compared with those water-soluble compounds which differ from them by one or more additional ionic groups. Water-insoluble is to be understood in the context of the invention as meaning those UV filters which at 20° C. dissolve to not more than 1% by weight, in particular to not more than 0.1% by weight, in water. Moreover, these compounds should be soluble in customary cosmetic oil components at room temperature to at least 0.1, in particular to at least 1%, by weight. The use of water-insoluble UV filters can therefore be preferential according to the invention.

According to a further embodiment of the invention, those UV filters are preferred which contain a cationic group, in particular a quaternary ammonium group. These UV filters have the general structure U-Q.

The structural component U here is a group absorbing UV rays. This group can in principle be derived from the known, abovementioned UV filters employable in the cosmetic field, in which a group, as a rule a hydrogen atom, of the UV filter is replaced by a cationic group Q, in particular with a quaternary amino function.

Compounds from which the structural component U can be derived are, for example,
substituted benzophenones,
p-aminobenzoic acid esters,
diphenylacrylic acid esters,
cinnamic acid esters,
salicylic acid esters,
benzimidazoles and
o-aminobenzoic acid esters.

Structural components U which are derived from cinnamamide or from N,N-dimethylaminobenzamide are preferred according to the invention.

The structural components U can in principle be chosen such that the absorption maximum of the UV filter can be either in the UVA (315-400 nm), or in the UVB (280-315 nm) or in the UVC (<280 nm) range. UV filters having an absorption maximum in the UVB range, in particular in the range from approximately 280 to approximately 300 nm, are particularly preferred.

Furthermore, the structural component U, also depending on the structural component Q, is preferably chosen such that the molar extinction coefficient of the UV filter at the absorption maximum is above 15 000, in particular above 20 000.

The structural component Q preferably contains as a cationic group a quaternary ammonium group. This quaternary ammonium group can in principle be connected directly to the structural component U, such that the structural component U is one of the four substituents of the positively charged nitrogen atom. Preferably, however, one of the four substituents on the positively charged nitrogen atom is a group, in particular an alkylene group having 2 to 6 carbon atoms, which functions as a link between the structural component U and the positively charged nitrogen atom.

Advantageously, the group Q has the general structure —(CH₂)ₓ—N⁺R¹R²R³X⁻, in which x is an integer from 1 to 4, R¹ and R² independently of one another are C₁₋₄-alkyl groups, R³ is a C₁₋₂₂-alkyl group or a benzyl group and X⁻ is a physiologically tolerable anion. In the context of this general structure, x is preferably the number 3, R¹ and R² are in each case a methyl group and R³ is either a methyl group or a saturated or unsaturated, linear or branched hydrocarbon chain having 8 to 22, in particular 10 to 18, carbon atoms.

Physiologically tolerable anions are, for example, inorganic anions, such as halides, in particular chloride, bromide and fluoride, sulfate ions and phosphate ions, and organic anions such as lactate, citrate, acetate, tartrate, methosulfate and tosylate.

Two preferred UV filters having cationic groups are the compounds cinnamic acid amidopropyltrimethylammonium chloride (Incroquat®UV-283) and dodecyldimethylaminobenzamidopropyldimethylammonium tosylate (Escalol® HP 610) obtainable as commercial products.

Of course, the teaching according to the invention also includes the use of a combination of several UV filters. In the context of this embodiment, the combination of at least one water-insoluble UV filter with at least one UV filter having a cationic group is preferred.

The UV filters (I) are present in the compositions used according to the invention customarily in amounts of from 0.1-5% by weight, based on the total composition. Amounts of from 0.4-2.5% by weight are preferred.

The action of the cationic starch derivatives (A) according to the invention can furthermore be increased by a 2-pyrrolidinone-5-carboxylic acid and its derivatives (J). A further subject of the invention is therefore the use of the ionic starch derivatives in combination with derivatives of 2-pyrrolidinone-5-carboxylic acid. The sodium, potassium, calcium, magnesium or ammonium salts are preferred in which the ammonium ion, in addition to hydrogen, carries one to three C₁- to C₄-alkyl groups. The sodium salt is very particularly preferred. The amounts employed in the compositions according to the invention are 0.05 to 10% by weight, based on the total composition, particularly preferably 0.1 to 5, and in particular 0.1 to 3% by weight.

The combination of the cationic starch derivatives (A) with the vitamins, provitamins and vitamin precursors, and their derivatives (K) has likewise proven advantageous.

Here, according to the invention those vitamins, provitamins and vitamin precursors are preferred which customarily are assigned to the groups A, B, C, E, F and H.

The group of the substances designated as vitamin A includes retinol (vitamin A₁), and 3,4-didehydroretinol (vitamin A₂). β-Carotene is the provitamin of retinol. According to the invention, a suitable vitamin A component is, for example, vitamin A acid and its esters, vitamin A aldehyde and vitamin A alcohol, and its esters such as the palmitate and the acetate. The preparations used according to the invention contain the vitamin A component preferably in amounts of from 0.05-1% by weight, based on the total preparation.

The vitamin B group or the vitamin B complex includes, inter alia
vitamin B₁ (thiamine)
vitamin B₂ (riboflavine)
vitamin B3. The compounds nicotinic acid and nicotinamide (niacinamide) are often included under this name. Nicotinamide, which is present in the compositions used according to the invention preferably in amounts of from 0.05 to 1% by weight, based on the total composition, is preferred according to the invention.
vitamin B₅ (pantothenic acid, panthenol and pantolactone). In the context of this group, panthenol and/or pantolactone is preferably employed. Derivatives of panthenol employable according to the invention are in particular the esters and ethers of panthenol, and cationically derivatized panthenols. Individual representatives are, for example, panthenol triacetate, panthenol monoethyl ether and its monoacetate, and the cationic panthenol derivatives disclosed in WO 92/13829. The compounds of the vitamin B₅ type mentioned are present in the compositions used according to the invention preferably in amounts of from 0.05-10% by weight, based on the total composition. Amounts of 0.1-5% by weight are particularly preferred.
vitamin B₆ (pyridoxine, and pyridoxamine and pyridoxal).

Vitamin C (ascorbic acid). Vitamin C is employed in the compositions used according to the invention preferably in amounts of from 0.1 to 3% by weight, based on the total composition. The use in the form of the palmitic acid ester, the glucosides or phosphates can be preferential. The use in combination with tocopherols can likewise be preferential.

Vitamin E (tocopherols, in particular α-tocopherol). Tocopherol and its derivatives, including, in particular, the esters such as the acetate, the nicotinate, the phosphate and the succinate, are present in the compositions used according to the invention preferably in amounts of 0.05-1% by weight, based on the total composition.

Vitamin F. The term “Vitamin F” is customarily understood as meaning essential fatty acids, in particular linoleic acid, linolenic acid and arachidonic acid.

Vitamin H. The compound (3aS,4S,6aR)-2-oxohexahydro-thienol[3,4-d]-imidazole-4-valeric acid is designated as vitamin H, for which, however, the trivial name biotin has meanwhile gained acceptance. Biotin is present in the compositions used according to the invention preferably in amounts of from 0.0001 to 1.0% by weight, in particular in amounts of from 0.001 to 0.01% by weight.

Preferably, the compositions used according to the invention contain vitamins, provitamins and vitamin precursors from groups A, B, E and H.

Panthenol, pantolactone, pyridoxine and its derivatives, and nicotinamide and biotin are particularly preferred.

Finally, the action of the cationic starch derivatives (A) can also be increased by combined use with plant extracts (L).

Customarily, these extracts are prepared by extraction of the whole plant. However, in individual cases it can also be preferential to prepare the extracts exclusively from the flowers and/or leaves of the plant.

With respect to the plant extracts utilizable according to the invention, reference is in particular made to the extracts which are mentioned in the table beginning on page 44 of the third edition of the guideline for the contents declaration of cosmetic compositions, published by the Industrieverband Körperpflege- and Waschmittel e.V. (IKW), Frankfurt.

According to the invention, the extracts of green tea, oak bark, stinging nettle, Hamamelis, hops, henna, camomile, burdock root, horsetail, hawthorn, linden blossom, almond, aloe vera, pine needle, horse chestnut, sandalwood, juniper, coconut, mango, apricot, lime, wheat, kiwi, melon, orange, grapefruit, sage, rosemary, birch, mallow, lady's smock, wild thyme, yarrow, thyme, melissa, restharrow, coltsfoot, hibiscus, meristem, ginseng and ginger root are especially preferred.

The extracts of green tea, oak bark, stinging nettle, Hamamelis, hops, camomile, burdock root, horsetail, linden blossom, almond, aloe vera, coconut, mango, apricot, lime, wheat, kiwi, melon, orange, grapefruit, thyme, rosemary, birch, lady's smock, wild thyme, yarrow, restharrow, meristem, ginseng and ginger root are particularly preferred.

The extracts of green tea, almond, aloe vera, coconut, mango, apricot, lime, wheat, kiwi and melon are very particularly suitable for the use according to the invention.

As extracting agents for the preparation of the plant extracts mentioned, water, alcohols and their mixtures can be used. Among the alcohols, lower alcohols such as ethanol and isopropanol, but in particular polyhydric alcohols such as ethylene glycol and propylene glycol, both as the sole extracting agent and as a mixture with water, are preferred here. Plant extracts based on water/propylene glycol in the ratio 1:10 to 10:1 have proven particularly suitable.

The plant extracts can be employed according to the invention either in pure form or in dilute form. If they are employed in dilute form, they customarily contain about 2-80% by weight of active substance and, as a solvent, the extracting agent or extracting agent mixture employed in their production.

Furthermore, it can be preferential in the compositions according to the invention to employ mixtures of several, in particular of two, different plant extracts.

Additionally, it can prove advantageous if, in addition to the cationic starch derivatives (A) according to the invention, penetration aids und/or swelling agents (M) are present. For this purpose, among these are to be included, for example, urea and urea derivatives, guanidine and its derivatives, arginine and its derivatives, sodium silicate, imidazole and its derivatives, histidine and its derivatives, benzyl alcohol, glycerol, glycol and glycol ethers, propylene glycol and propylene glycol ethers, for example propylene glycol monoethyl ether, carbonates, hydrogencarbonates, diols and triols, and in particular 1,2-diols and 1,3-diols such as, for example, 1,2-propanediol, 1,2-pentanediol, 1,2-hexanediol, 1,2-dodecanediol, 1,3-propanediol, 1,6-hexanediol, 1,5-pentanediol, 1,4-butanediol.

Advantageously, within the meaning of the invention short-chain carboxylic acids (N) can additionally assist the cationic starch derivatives (A) Short-chain carboxylic acids and their derivatives within the meaning of the invention are understood as meaning carboxylic acids which can be saturated or unsaturated and/or straight-chain or branched or cyclic and/or aromatic and/or heterocyclic and have a molecular weight of less than 750. Within the meaning of the invention, saturated or unsaturated straight-chain or branched carboxylic acids having a chain length of 1 up to 16 carbon atoms in the chain can be preferential, those having a chain length of 1 up to 12 carbon atoms in the chain are very particularly preferred.

The short-chain carboxylic acids within the meaning of the invention can have one, two, three or more carboxyl groups. Within the meaning of the invention, carboxylic acids having several carboxyl groups are preferred, in particular di- and tricarboxylic acids. The carboxy groups can be present completely or partially as the ester, acid anhydride, lactone, amide, imido acid, lactam, lactim, dicarboximide, carbohydrazide, hydrazone, hydroxam, hydroxime, amidine, amidoxime, nitrile, phosphonic or phosphate ester. The carboxylic acids according to the invention can, of course, be substituted along the carbon chain or the ring structure. The substituents of the carboxylic acids according to the invention include, for example C₁-C₈-alkyl, C₂-C₈-alkenyl, aryl, aralkyl and aralkenyl, hydroxymethyl, C₂-C₈-hydroxyalkyl, C₂-C₈-hydroxy-alkenyl, aminomethyl, C₂-C₈-aminoalkyl, cyaho, formyl, oxo, thioxo, hydroxy, mercapto, amino, carboxy or imino groups. Preferred substituents are C₁-C₈-alkyl, hydroxymethyl, hydroxy, amino and carboxy groups. Substituents in the α-position are particularly preferred. Very particularly preferred substituents are hydroxy, alkoxy and amino groups, where the amino function can optionally be further substituted by alkyl, aryl, aralkyl and/or alkenyl radicals. Furthermore, the phosphonic and phosphate esters are likewise preferred carboxylic acid derivatives.

Examples of carboxylic acids according to the invention which may be mentioned are formic acid, acetic acid, propionic acid, butyric acid, isobutyric acid, valeric acid, isovaleric acid, pivalic acid, oxalic acid, malonic acid, succinic acid, glutaric acid, glyceric acid, glyoxylic acid, adipic acid, pimelic acid, suberic acid, azelaic acid, sebacic acid, propiolic acid, crotonic acid, isocrotonic acid, elaidic acid, maleic acid, fumaric acid, muconic acid, citraconic acid, mesaconic acid, camphoric acid, benzoic acid, o,m,p-phthalic acid, naphthoic acid, toluic acid, hydratropic acid, atropic acid, cinnamic acid, isonicotinic acid, nicotinic acid, bicarbamic acid, 4,4′-dicyano-6,6′-binicotinic acid, 8-carbamoyl-octanoic acid, 1,2,4-pentanetricarboxylic acid, 2-pyrrolecarboxylic acid, 1,2,4,6,7-naphthalene-pentaacetic acid, malonaldehydic acid, 4-hydroxy-phthalamic acid, 1-pyrazolecarboxylic acid, gallic acid or propanetricarboxylic acid, a dicarboxylic acid selected from the group which is formed by compounds of the general formula (N-I), 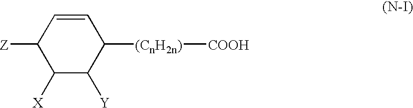
in which Z is a linear or branched alkyl or alkenyl group having 4 to 12 carbon atoms, n is a number from 4 to 12 and one of the two groups X and Y is a COOH group and the other is hydrogen or a methyl or ethyl radical, dicarboxylic acids of the general formula (N-I), which additionally also carry 1 to 3 methyl or ethyl substituents on the cyclohexene ring, and dicarboxylic acids which are formed from the dicarboxylic acids according to formula (N-I) formally by addition of a molecule of water to the double bond in the cyclohexene ring.

Dicarboxylic acids of the formula (N-I) are known in the literature.

A preparation process can be taken, for example, from U.S. Pat. No. 3,753,968. German patent 22 50 055 discloses the use of these dicarboxylic acids in liquid soap compositions. From German laid-open specification 28 33 291, deodorants are known which contain zinc or magnesium salts of these dicarboxylic acids. Finally, from German laid-open specification 35 03 618, compositions for washing and rinsing the hair are known in which, by addition of these dicarboxylic acids, a noticeably improved hair-cosmetic action of the water-soluble ionic polymers contained in the composition is obtained. Finally, from German laid-open specification 197 54 053, compositions for hair treatment are known which have care effects.

The dicarboxylic acids of the formula (N-I) can be prepared, for example, by reaction of polyunsaturated dicarboxylic acids with unsaturated monocarboxylic acids in the form of a Diels-Alder cyclization. Customarily, the process will start out from a polyunsaturated fatty acid as the dicarboxylic acid component. Linoleic acid, which is accessible from natural fats and oils, is preferred. As a monocarboxylic acid component, in particular acrylic acid, but also, for example, methacrylic acid and crotonic acid, are preferred. Customarily, in reactions according to Diels-Alder, isomer mixtures are formed in which one component is present in an excess. These isomer mixtures can be employed according to the invention just like the pure compounds.

Also employable according to the invention in addition to the preferred dicarboxylic acids according to formula (N-I) are those dicarboxylic acids which differ from the compounds according to formula (N-I) by 1 to 3 methyl or ethyl substituents on the cyclohexyl ring or are formed from these compounds formally by addition of one molecule of water to the double bond of the cyclohexene ring.

The dicarboxylic acid (mixture) which is obtainable by reaction of linoleic acid with acrylic acid has proven particularly effective according to the invention. This is a mixture of 5- and 6-carboxy-4-hexyl-2-cyclohexene-1-octanoic acid. Such compounds are commercially obtainable under the names Westvaco Diacid® 1550 and Westvaco Diacid® 1595 (manufacturer: Westvaco).

In addition to the short-chain carboxylic acids according to the invention mentioned above by way of example themselves, their physiologically tolerable salts can also be employed according to the invention. Examples of such salts are the alkali metal salts, alkaline earth metal salts, zinc salts, and ammonium salts, under which in the context of the present application the mono-, di- and trimethyl-, ethyl- and hydroxyethylammonium salts are also to be understood. Very particularly preferably, in the context of the invention, however, acids neutralized with alkaline-reacting amino acids, such as, for example, arginine, lysine, ornithine and histidine, can be employed. Furthermore, it can be preferential for formulation reasons to select the carboxylic acid from the water-soluble representatives, in particular the water-soluble salts.

Furthermore, it is preferred according to the invention to employ hydroxycarboxylic acids and, here in turn, in particular the dihydroxy-, trihydroxy- and polyhydroxy-carboxylic acids, and the dihydroxy-, trihydroxy- and polyhydroxydi-, tri- and polycarboxylic acids together with the cationic starch derivatives (A). It has been shown here that, in addition to the hydroxycarboxylic acids, the hydroxycarboxylic acid esters, and the mixtures of hydroxycarboxylic acids and their esters and also polymeric hydroxycarboxylic acids and their esters can be very particularly preferential. Preferred hydroxycarboxylic acid esters are, for example, full esters of glycolic acid, lactic acid, malic acid, tartaric acid or citric acid. Further fundamentally suitable hydroxycarboxylic acid esters are esters of β-hydroxypropionic acid, of tartronic acid, of D-gluconic acid, of saccharic acid, of mucic acid or of glucuronic acid. Suitable alcohol components of these esters are primary, linear or branched aliphatic alcohols having 8-22 carbon atoms, that is, for example, fatty alcohols or synthetic fatty alcohols. In this context the esters of C₁₂-C₁₅-fatty alcohols are particularly preferred. Esters of this type are commercially obtainable, e.g. under the trademark Cosmacol® from EniChem, Augusta Industriale. Particularly preferred polyhydroxypolycarboxylic acids are polylactic acid and polytartaric acid, and their esters.

Finally, it is, of course, also possible in the context of the teaching of the invention in addition to the cationic starch derivatives (A) according to the invention to use protein hydrolyzates and their derivatives (P) which are not derived from natural silk.

Protein hydrolyzates are product mixtures which are obtained by acid-, base- or enzyme-catalyzed degradation of proteins. The term protein hydrolyzates is understood according to the invention as also meaning total hydrolyzates, and individual amino acids and their derivatives, and mixtures of various amino acids. Furthermore, according to the invention polymers synthesized from amino acids and amino acid derivatives are understood under the term protein hydrolyzates. Polyalanine, polyasparagine, polyserine etc., for example, are to be counted among the latter. Further examples of compounds employable according to the invention are L-alanyl-L-proline, polyglycine, glycyl-L-glutamine or D/L-methionine-5-methylsulfonium chloride. Of course, according to the invention β-amino acids and their derivatives such as β-alanine, anthranilic acid or hippuric acid can also be employed. The molecular weight of the protein hydrolyzates employable according to the invention is between 75, the molecular weight for glycine, and 200 000, preferably the molecular weight is 75 to 50 000 and very particularly preferably 75 to 20 000 daltons.

According to the invention, protein hydrolyzates both of vegetable and animal or marine or synthetic origin can be employed.

Animal protein hydrolyzates are, for example, elastin, collagen, keratin and lactoprotein hydrolyzates, which can also be present in the form of salts. Such products are marketed, for example, under the trademarks Dehylan® (Cognis), Promoise (Interorgana), Collapuron® (Cognis), Nutrilan® (Cognis), Gelita-Sol® (Deutsche Gelatine Fabriken Stoess & Co), Lexein® (Inolex) and Kerasol® (Croda).

According to the invention, the use of protein hydrolyzates of vegetable origin is preferred, e.g. soybean, almond, pea, potato and wheat protein hydrolyzates. Such products are obtainable, for example, under the trademarks Gluadin® (Cognis), DiaMin® (Diamalt), Lexein® (Inolex), Hydrosoy® (Croda), Hydrolupin® (Croda), Hydrosesame® (Croda), Hydrotritium® (Croda) and Crotein® (Croda).

Even though the use of the protein hydrolyzates as such is preferred, amino acid mixtures obtained in other ways can optionally also be employed in their place. Likewise, the use of derivatives of protein hydrolyzates is possible, for example in the form of their fatty acid condensation products. Such products are marketed, for example, under the names Lamepon® (Cognis), Lexein® (Inolex), Crolastin® (Croda) or Crotein® (Croda).

Of course, the teaching according to the invention comprises all isomeric forms, such as cis isomers, trans isomers, diastereomers and chiral isomers.

According to the invention, it is also possible to employ a mixture of several protein hydrolyzates (P).

The protein hydrolyzates (P) are present in the compositions in concentrations of from 0.01% by weight up to 20% by weight, preferably from 0.05% by weight up to 15% by weight and very particularly preferably in amounts from 0.05% by weight up to 5% by weight.

The starch derivatives (A) according to the invention can in principle be added directly to the colorant, the waving agent or the setting agent. The application of the ionic starch derivatives according to the invention to the keratinic fibers can, however, also be carried out in a separate step, either before or following the actual dyeing or waving process. Separate treatments, optionally even days or weeks before or after the hair treatment, for example by dyeing or waving, are also included by the teaching according to the invention. Preferably, however, the application of the cationic starch derivatives according to the invention can take place after the appropriate hair treatment, such as dyeing or waving, in particular in the appropriate hair treatment compositions.

The term dyeing process in this case comprises all processes known to the person skilled in the art in which a colorant is applied to the hair, which is optionally dampened, and this colorant is either left on the hair for a time of between a few minutes and about 45 minutes and subsequently rinsed out with water or a surfactant-containing composition or is left entirely on the hair. Reference is made in this connection expressly to the known monographs, e.g. K. H. Schrader, Grundlagen und Rezepturen der Kosmetika [Bases and formulations of cosmetics], 2nd edition, Huthig Buch Verlag, Heidelberg, 1989, which reproduce the corresponding knowledge of the person skilled in the art.

The term waving process here includes all processes known to the person skilled in the art in which a waving agent is applied to the hair, which is optionally dampened and wound on curlers, and this agent is left on the hair for a time of between a few minutes and about 45 minutes and subsequently rinsed out with water or a surfactant-containing composition, subsequently a permanent wave setting lotion is applied to the hair and this lotion is left on the hair for a time of between a few minutes and about 45 minutes and subsequently rinsed out with water or a surfactant-containing composition. Reference is made in this connection expressly to the known monographs, e.g. K. H. Schrader, Grundlagen and Rezepturen der Kosmetika, 2nd edition, Huthig Buch Verlag, Heidelberg, 1989, which reproduce the corresponding knowledge of the person skilled in the art.

With respect to the manner according to which the cationic starch derivatives according to the invention are applied to the keratinic fibers, in particular human hair, no fundamental restrictions exist. Suitable formulations for these preparations are, for example, creams, lotions, solutions, waters, emulsions such as W/O, O/W, PIT emulsions (emulsions according to the teaching of phase inversion, called PIT), microemulsions and multiple emulsions, gels, sprays, aerosols and foaming aerosols. The pH of these preparations can in principle be 2-11. It is preferably between 5 and 11, values from 6 to 10 being particularly preferred. For the adjustment of this pH, virtually any acid or base utilizable for cosmetic purposes can be used. Preferred bases are ammonia, alkali metal hydroxides, monoethanolamine, triethanolamine, and N,N,N′,N′-tetrakis-(2-hydroxypropyl)ethylenediamine.

Preparations remaining on the hair have proven effective and can therefore be preferred embodiments of the teaching according to the invention. Remaining on the hair is understood according to the invention as meaning those preparations which are not rinsed out of the hair again after a period of a few seconds up to one hour with the aid of water or an aqueous solution in the course of the treatment. Rather, the preparations remain on the hair until the next hair wash, i.e. as a rule more than 12 hours.

According to a second preferred embodiment, these preparations are formulated as a hair tonic or hair conditioner. The preparations according to the invention according to this embodiment can be rinsed out with water or an at least mainly water-containing composition after passage of this time of action; they can, however, be left on the hair, as explained in detail above. It can be preferential here to apply the preparation according to the invention to the hair before the application of a cleansing agent, a waving agent or other hair treatment agents. In this case, the preparation according to the invention serves as a structural protection for the subsequent applications.

According to a further preferred embodiment, the compositions according to the invention can, however, for example, also be cleansing agents such as shampoos, caring agents, such as rinses, setting compositions such as hair setting agents, foam setting agents, styling gels and hairdryer waves, permanent shaping agents such as permanent wave and setting agents, and in particular pretreatment agents or after rinses employed in the course of a permanent wave process or dyeing process.

In addition to the absolutely necessary cationic starch derivatives (A) according to the invention and the further, abovementioned preferred components, these preparations can in principle contain all further components known to the person skilled in the art for such cosmetic compositions.

Further active ingredients, excipients and additives are, for example,
nonionic polymers such as, for example, vinylpyrrolidone/vinyl acrylate copolymers, polyvinyl-pyrrolidone and vinylpyrrolidone/vinyl acetate copolymers and polysiloxanes,
thickening agents such as agar-agar, guar gum, alginates, xanthan gum, gum arabic, Karaya gum, carob bean flour, linseed gums, dextrans, cellulose derivatives, e.g. methylcellulose, hydroxyalkylcellulose and carboxymethylcellulose, starch fractions and derivatives such as amylose, amylopectin and dextrins, clays such as, for example, bentonite or fully synthetic hydrocolloids such as, for example polyvinyl alcohol,
hair-conditioning compounds such as phospholipids, for example soybean lecithin, egg lecithin and cephalins, and also silicone oils,
perfume oils, dimethyl isosorbide and cyclodextrins,
solvents and solubilizers such as ethanol, isopropanol, ethylene glycol, propylene glycol, glycerol and diethylene glycol,
symmetrical and unsymmetrical, linear and branched dialkyl ethers having a total of between 12 and 36 carbon atoms, in particular 12 to 24 carbon atoms, such as, for example, di-n-octyl ether, di-n-decyl ether, di-n-nonyl ether, di-n-undecyl ether and di-n-dodecyl ether, n-hexyl-n-octyl ether, n-octyl-n-decyl ether, n-decyl-n-undecyl ether, n-undecyl-n-dodecyl ether and n-hexyl-n-undecyl ether, and di-tert-butyl ether, diisopentyl ether, di-3-ethyldecyl ether, tert-butyl-n-octyl ether, isopentyl-n-octyl ether and 2-methylpentyl-n-octyl ether,
fatty alcohols, in particular linear and/or saturated fatty alcohols having 8 to 30 carbon atoms,
monoesters of C₈ to C₃₀-fatty acids with alcohols having 6 to 24 carbon atoms,
fiber structure-improving active ingredients, in particular mono-, di- and oligosaccharides, such as for example glucose, galactose, fructose, levulose and lactose,
conditioning active ingredients such as paraffin oils, vegetable oils, e.g. sunflower oil, orange oil, almond oil, wheatgerm oil and peach kernel oil, and
phospholipids, for example soybean lecithin, egg lecithin and cephalins,
quaternized amines such as methyl-1-alkylamidoethyl-2-alkylimidazolinium methosulfate,
antifoams such as silicones,
dyes for coloring the composition,
antidandruff active ingredients such as piroctone olamine, zinc omadine and climbazole,
active ingredients such as allantoin and bisabolol, cholesterol,
consistency-imparting agents such as sugar esters, polyol esters or polyol alkyl ethers,
fats and waxes such as spermaceti, beeswax, montan wax and paraffins,
fatty acid alkanolamides,
complexing agents such as EDTA, NTA, β-alaninediacetic acid and phosphonic acids, swelling agents and penetrants such as primary, secondary and tertiary phosphates,
opacifying, agents such as latex, styrene/PVP and styrene/acrylamide copolymers
pearl luster agents such as ethylene glycol mono- and distearate, and PEG-3 distearate,
pigments,
reductants such as, for example, thioglycolic acid and its derivatives, thiolactic acid, cysteamine, thiomalic acid and α-mercaptoethanesulfonic acid,
propellants such as propane-butane mixtures, N₂O, dimethyl ether, CO₂ and air,
antioxidants.

With respect to further optional components, and the amounts of these components employed, reference is expressly made to the relevant handbooks known to the person skilled in the art, e.g. the abovementioned monograph by K. H. Schrader.

In a fourth embodiment of the teaching according to the invention, it can be preferential to incorporate the ionic starch derivatives (A) directly into colorants or tinting agents, that is to employ the cationic starch derivatives (A) according to the invention into combinations with dyes and/or dye precursors.

As such, oxidation dye precursors of the developer (B1) and coupler type (B2), natural and synthetic direct dyes (C) and precursors of naturally analogous dyes, such as indole and indoline derivatives, and mixtures of representatives of one or more of these groups can be employed.

As oxidation dye precursors of the developer type (B1), primary aromatic amines having a further free or substituted hydroxy or amino group situated in the para- or ortho-position, diaminopyridine derivatives, heterocyclic hydrazones, 4-aminopyrazole derivatives, and 2,4,5,6-tetraminopyrimidine and its derivatives are customarily employed. Suitable developer components are, for example, p-phenylendiamine, p-toluylenediamine, p-aminophenol, o-aminophenol, 1-(2′-hydroxyethyl)-2,5-diaminobenzene, N,N-bis-(2-hydroxyethyl)-p-phenylendiamine, 2-(2,5-diamino-phenoxy)ethanol, 4-amino-3-methylphenol, 2,4,5,6-tetra-aminopyrimidine, 2-hydroxy-4,5,6-triaminopyrimidine, 4-hydroxy-2,5,6-triaminopyrimidine,2,4-dihydroxy-5,6-diaminopyrimidine, 2-dimethylamino-4,5,6-triamino-pyrimidine, 2-hydroxymethylamino-4-aminophenol, bis(4-aminophenyl)amine, 4-amino-3-fluorophenol, 2-aminomethyl-4-aminophenol, 2-hydroxymethyl-4-aminophenol, 4-amino-2-((diethylamino)methyl)phenol, bis(2-hydroxy-5-aminophenyl)methane, 1,4-bis(4-aminophenyl)diazacycloheptane, 1,3-bis(N-(2-hydroxyethyl)-N-(4-aminophenylamino))-2-propanol, 4-amino-2-(2-hydroxyethoxy)phenol, 1,10-bis(2,5-diaminophenyl)-1,4,7,10-tetraoxadecane, and 4,5-diaminopyrazole derivatives according to EP 0 740 931 and WO 94/08970 such as, for example, 4,5-diamino-1-(2′-hydroxyethyl)pyrazole. Particularly advantageous developer components are p-phenylendiamine, p-toluylenediamine, p-aminophenol, 1-(2′-hydroxyethyl)-2,5-diaminobenzene, 4-amino-3-methylphenol, 2-aminomethyl-4-aminophenol, 2,4,5,6-tetraminopyrimidine, 2-hydroxy-4,5,6-triamino-pyrimidine, 4-hydroxy-2,5,6-triaminopyrimidine.

As oxidation dye precursors of the coupler type (B2), as a rule m-phenylenediamine derivatives, naphthols, resorcinol and resorcinol derivatives, pyrazolone and m-aminophenol derivatives are used. Examples of such coupler components are m-aminophenol and its derivatives such as, for example, 5-amino-2-methylphenol, 5-(3-hydroxypropylamino)-2-methylphenol, 3-amino-2-chloro-6-methylphenol, 2-hydroxy-4-amino-phenoxyethanol, 2,6-dimethyl-3-aminophenol, 3-trifluoroacetylamino-2-chloro-6-methylphenol, 5-amino-4-chloro-2-methylphenol, 5-amino-4-methoxy-2-methylphenol, 5-(2′-hydroxyethyl)amino-2-methylphenol, 3-(diethylamino)phenol, N-cyclopentyl-3-aminophenol, 1,3-dihydroxy-5-(methylamino)benzene, 3-(ethylamino)-4-methylphenol and 2,4-dichloro-3-aminophenol, o-aminophenol and its derivatives, m-diaminobenzene and its derivatives such as, for example, 2,4-diaminophenoxyethanol, 1,3-bis(2,4-diaminophenoxy)propane, 1-methoxy-2-amino-4-(2′-hydroxyethyl amino)benzene, 1,3-bis (2,4-diaminophenyl)propane, 2,6-bis(2-hydroxyethylamino)-1-methylbenzene and 1-amino-3-bis(2′-hydroxyethyl)aminobenzene, o-diaminobenzene and its derivatives such as, for example, 3,4-diaminobenzoic acid and 2,3-diamino-1-methylbenzene, di- and trihydroxybenzene derivatives such as for example resorcinol, resorcinol monomethyl ether, 2-methylresorcinol, 5-methylresorcinol, 2,5-dimethylresorcinol, 2-chlororesorinol, 4-chloro-resorcinol, pyrogallol and 1,2,4-trihydroxybenzene, pyridine derivatives such as, for example, 2,6-dihydroxypyridine, 2-amino-3-hydroxypyridine, 2-amino-5-chloro-3-hydroxypyridine, 3-amino-2-methylamino-6-methoxypyridine, 2,6-dihydroxy-3,4-dimethylpyridine, 2,6-dihydroxy-4-methylpyridine, 2,6-diaminopyridine, 2,3-diamino-6-methoxypyridine and 3,5-diamino-2,6-dimethoxypyridine, naphthalene derivatives such as, for example, 1-naphthol, 2-methyl-1-naphthol, 2-hydroxymethyl-1-naphthol, 2-hydroxyethyl-1-naphthol, 1,5-dihydroxynaphthalene, 1,6-dihydroxynaphthalene, 1,7-dihydroxynaphthalene, 1,8-dihydroxynaphthalene, 2,7-dihydroxynaphthalene and 2,3-dihydroxynaphthalene, morpholine derivatives such as, for example, 6-hydroxybenzomorpholine and 6-aminobenzomorpholine, quinoxaline derivatives such as, for example, 6-methyl-1,2,3,4-tetrahydroquinoxaline, pyrazole derivatives such as, for example, 1-phenyl-3-methylpyrazol-5-one, indole derivatives such as, for example, 4-hydroxyindole, 6-hydroxyindole and 7-hydroxyindole, methylenedioxybenzene derivatives such as, for example, 1-hydroxy-3,4-methylenedioxybenzene, 1-amino-3,4-methylenedioxybenzene and 1-(2′-hydroxyethyl)amino-3,4-methylenedioxybenzene.

Particularly suitable coupler components are 1-naphthol, 1,5-, 2,7- and 1,7-dihydroxynaphthalene, 3-aminophenol, 5-amino-2-methylphenol, 2-amino-3-hydroxypyridine, resorcinol, 4-chlororesorcinol, 2-chloro-6-methyl-3-aminophenol, 2-methylresorcinol, 5-methylresorcinol, 2,5-dimethylresorcinol and 2,6-dihydroxy-3,4-dimethylpyridine.

Direct dyes are customarily nitrophenylenediamines, nitroaminophenols, azo dyes, anthraquinones or indophenols. Particularly suitable direct dyes are the compounds known under the international names or trade names HC Yellow 2, HC Yellow 4, HC Yellow 5, HC Yellow 6, Basic Yellow 57, Disperse Orange 3, HC Red 3, HC Red BN, Basic Red 76, HC Blue 2, HC Blue 12, Disperse Blue 3, Basic Blue 99, HC Violet 1, Disperse Violet 1, Disperse Violet 4, Disperse Black 9, Basic Brown 16 and Basic Brown 17, and 1,4-bis(β-hydroxyethyl)amino-2-nitrobenzene, 4-amino-2-nitrodiphenylamine-2′-carboxylic acid, 6-nitro-1,2,3,4-tetrahydro-quinoxaline, hydroxyethyl-2-nitrotoluidine, picramic acid, 2-amino-6-chloro-4-nitrophenol, 4-ethylamino-3-nitrobenzoic acid and 2-chloro-6-ethylamino-1-hydroxy-4-nitrobenzene.

Direct dyes occurring in nature are, for example, henna red, henna neutral, camomile flower, sandalwood, black tea, alder buckthorn bark, sage, logwood, madder root, Catechu, Sedre and alkanna root.

It is not necessary that the oxidation dye precursors or the direct dyes are in each case homogeneous compounds. Rather, still further components can be present in minor amounts in the hair colorants according to the invention, due to the preparation processes for the individual dyes, provided this does not influence the color result disadvantageously or have to be excluded for other reasons, e.g. toxicological reasons.

With respect to the dyes employable in the hair colorants and tinting agents according to the invention, reference is furthermore made expressly to the monograph Ch. Zviak, The Science of Hair Care, chapter 7 (pages 248-250; direct dyes), and chapter 8, pages 264-267; oxidation dye precursors), published as volume 7 of the series “Dermatology” (ed.: Ch. Culnan and H. Maibach), Verlag Marcel Dekker Inc., New York, Basle, 1986, and the “Europäische Inventar der Kosmetik-Rohstoffe”, published by the European Community, obtainable in diskette form from the Bundesverband Deutscher Industrie- and Handelsunternehmen für Arzneimittel, Reformwaren and Körperpflegemittel e.V., Mannheim.

The precursors of naturally analogous dyes used are, for example, indoles and indolines, and their physiologically tolerable salts. Preferably, those indoles and indolines are employed which contain at least one hydroxy or amino group, preferably as a substituent on the six-membered ring. These groups can carry further substituents, e.g. in the form of an etherification or esterification of the hydroxy group or an alkylation of the amino group. 5,6-Dihydroxyindoline, N-methyl-5,6-dihydroxyindoline, N-ethyl-5,6-dihydroxyindoline, N-propyl-5,6-dihydroxy-indoline, N-butyl-5,6-dihydroxyindoline, 5,6-dihydroxy-indoline-2-carboxylic acid, 6-hydroxyindoline, 6-aminoindoline and 4-aminoindoline, and 5,6-di-hydroxyindole, N-methyl-5,6-dihydroxyindole, N-ethyl-5,6-dihydroxyindole, N-propyl-5,6-dihydroxyindole, N-butyl-5,6-dihydroxyindole, 5,6-dihydroxyindole-2-carboxylic acid, 6-hydroxyindole, 6-aminoindole and 4-aminoindole have particularly advantageous properties.

Particularly to be emphasized within this group are N-methyl-5,6-dihydroxyindoline, N-ethyl-5,6-dihydroxy-indoline, N-propyl-5,6-dihydroxyindoline, N-butyl-5,6-dihydroxyindoline and in particular 5,6-dihydroxyindoline, and N-methyl-5,6-dihydroxyindole, N-ethyl-5,6-dihydroxyindole, N-propyl-5,6-dihydroxy-indole, N-butyl-5,6-dihydroxyindole, and in particular 5,6-dihydroxyindole.

The indoline and indole derivatives in the colorants employed in the course of the process according to the invention are employed both as the free bases and in the form of their physiologically tolerable salts with inorganic or organic acids, e.g. the hydrochlorides, the sulfates and hydrobromides.

When using dye precursors of the indoline or indole type, it can be preferential to employ these together with at least one amino acid and/or least one oligopeptide. Preferred amino acids and aminocarboxylic acids are in particular α-aminocarboxylic acids and ω-aminocarboxylic acids. Among the α-aminocarboxylic acids, arginine, lysine, ornithine and histidine are in turn particularly preferred. A very particularly preferred amino acid is arginine, in particular in free form, but also employed as the hydrochloride.

Both the oxidation dye precursors and the direct dyes and the precursors of naturally analogous dyes are present in the compositions according to the invention preferably in amounts of from 0.01 to 20% by weight, preferably 0.1 to 5% by weight, in each case based on the total composition.

Hair colorants, in particular if the dyeing takes place oxidatively, be it with atmospheric oxygen or with other oxidants such as hydrogen peroxide, are customarily weakly acidic to alkaline, i.e. adjusted to pHs in the range from approximately 5 to 11. For this purpose, the colorants contain alkalizing agents, customarily alkali metal or alkaline earth metal hydroxides, ammonia or organic amines. Preferred alkalizing agents are monoethanolamine, monoisopropanolamine, 2-amino-2-methylpropanol, 2-amino-2-methyl-1,3-propanediol, 2-amino-2-ethyl-1,3-propanediol, 2-amino-2-methylbutanol and triethanolamine, and alkali metal and alkaline earth metal hydroxides. In particular monoethanolamine, triethanolamine, and 2-amino-2-methylpropanol and 2-amino-2-methyl-1,3-propanediol are preferred in the context of this group. The use of ω-amino acids such as ω-aminocaproic acid as an alkalizing agent is also possible.

If the formation of the actual hair dyes takes place in the course of an oxidative process, customary oxidants, such as, in particular, hydrogen peroxide or its addition products to urea, melamine or sodium borate can be used. Oxidation using atmospheric oxygen as the sole oxidant can, however, be preferential. Furthermore, it is possible to carry out the oxidation with the aid of enzymes, the enzymes being employed both for the production of oxidizing per compounds and for increasing the action of a small amount of oxidant present, or alternatively enzymes are used which transfer electrons from suitable developer components (reductants) to atmospheric oxygen. Oxidases such as tyrosinase, ascorbate oxidase and laccase but also glucose oxidase, uricase or pyruvate oxidase are preferred here. Furthermore, the procedure of increasing the action of small amounts (e.g. 1% and less, based on the total composition) of hydrogen peroxide by peroxidases may be mentioned.

Expediently, the preparation of the oxidant is then mixed with the dye precursors immediately before the dyeing of the hair with the preparation. The ready-to-use hair dyeing preparation resulting in this way should preferably have a pH in the range from 6 to 10. The application of the hair colorants in a weakly alkaline medium is particularly preferred. The application temperatures can be in the range between 15 and 40° C., preferably at the temperature of the scalp. After a time of action of about 5 to 45, in particular 15 to 30, minutes, the hair colorant is removed by rinsing out from the hair to be colored. Rewashing with a shampoo is unnecessary if a strongly surfactant-containing carrier, e.g. a coloring shampoo, was used.

In particular in the case of poorly dyeable hair, the preparation can be applied to the hair with the dye precursors without prior mixing with the oxidation component. After a time of action of 20 to 30 minutes, the oxidation component is then applied-optionally after an intermediate rinse. After a further time of action of 10 to 20 minutes, it is then rinsed and, if desired, reshampooed. In this embodiment, according to a first variant in which the prior application of the dye precursors is intended to bring about a better penetration into the hair, the appropriate composition is adjusted to a pH of approximately 4 to 7. According to a second variant, an atmospheric oxidation is first aimed at, the composition applied preferably having a pH of 7 to 10. In the subsequent accelerated reoxidation, the use of acidically adjusted peroxydisulfate solutions as oxidants can be preferential.

Furthermore, the formation of the dyeing can be assisted and increased by adding certain metal ions to the composition. Such metal ions are, for example, Zn²⁺ Cu²⁺, Fe²⁺, Fe³⁺, Mn²⁺, Mn⁴⁺, Li⁺, Mg²⁺, Ca²+ and Al³⁺. Zn²⁺, Cu²⁺ and Mn²⁺ are particularly suitable here. The metal ions can in principle be employed in the form of any desired, physiologically tolerable salt. Preferred salts are the acetates, sulfates, halides, lactates and tartrates. By use of these metals, both the formation of the dyeing can be accelerated and the color shade can be specifically influenced.

The invention relates fifthly to cosmetic compositions comprising:
a. the cationic starch derivatives (A)
b. and a compound selected from the group consisting of the surfactants (E) and/or the polymers (G).

With respect to further components of these compositions, reference is made to what has been stated above.

The invention relates sixthly to a process for the treatment of skin or hair, in which a composition comprising the cationic starch derivatives (A) according to the invention, as used in one of claims 1 to 7, is applied to the fibers, the composition being rinsed out again, if desired, after a time of action of 1 to 45 minutes.

### EXAMPLES

All quantitative data are, if not stated otherwise, parts by weight.

1. Demonstration of Action

Description of the Experimental Procedure:

Materials Used:

Test strands of hair: buffalo stomach hair from Kerling in a length of about 0.06 m, bound, left natural, white and washed.
Test hair dye: IGORA Fleur, shade 5-88 Burgundy from Schwarzkopf
   Treatment of the Test Strands of Hair:

The strands of hair are dyed for 30 minutes according to the use instructions using the hair dye IGORA Fleur, shade 5-88. After rinsing the strands of hair with lukewarm tap water, the strands of hair are dried. After a rest period (conditioning) of 24 h at 50% relative atmospheric humidity and 23° C., the respective aqueous test solutions are applied to the dry strands of hair in a liquor ratio of 1:1. The test solutions in each case contain 1.0% by weight of the respective ionic starch derivative. In the case of the reference sample, no ionic starch derivatives is present. The test solutions are lightly massaged into the strands of hair by hand. After a time of action of 5 minutes, the strands of hair are again rinsed out under running lukewarm tap water. After drying the strands of hair, these are again conditioned for 24 h. The LAB starting values are now measured. The strands of hair are then washed in an ultrasonic bath for 15 minutes with a 1.0% solution (active substance) of Texapon® NSO (Cognis) having a pH of 5.5. After rinsing out the strands with lukewarm tap water, the strands of hair are again dried and conditioned. Finally, the LAB values of the strands of hair are measured using the color measurement apparatus. For the measurement with the LAB color measurement apparatus, in each case 4 measurment points (2 on the front of the strand and 2 on the back of the strand) are measured before and after the treatment. The individual measurements must not differ by more than 2 here. The measurements are in each case combined to give mean values. For the calculation of the delta-E values, the LAB mean values of the same strand before and after treatment are used. The calculation is carried out according to the formula:
ΔE=((ΔL)²+(Δa)²+(Δb)²)^{0.5}
where L=luminosity, a=red component and b=blue component.

For the determination of the combabilities in the wet and dry hair, the strands of hair are combed out with the aid of a comb and comparatively assessed in comparison with the standard strand of hair. The subjective assessment extends from markedly better through better, identical, poorer to markedly poorer.

In table 1, the delta-E values for various ionic starch derivatives are juxtaposed in comparison with the standard (without starch derivatives). The results of the combabilities are indicated at the same time.

| | | Wet | Dry |
|---|---|---|---|
| | Delta-E value | combability | combability |
| Reference | 12.01 | | |
| (Nullwert) | | | |
| CI 50 A 40 | 12.21 | Markedly | Better |
| | | better | |
| CI 25 MO | 12.14 | Markedly | Markedly |
| | | better | better |
| CI 25 TA | 13.31 | Markedly | Markedly |
| | | better | better |
| HP CI 50 | 13.01 | Markedly | Markedly |
| | | better | better |

2. Application Examples

1. Hair Rinse

| | |
|---|---|
| Eumulgin ® B2¹ | 0.3 |
| Cetyl/stearyl alcohol | 3.3 |
| Sericin | 0.3 |
| Promois ® Silk-1000 | 0.7 |
| Isopropyl myristate | 0.5 |
| Lamesoft ® PO 65⁴ | 0.5 |
| Cat. rice starch, (MW 10000000 D, | 1.5 |
| Cationic degree of substitution 0.25) | |
| Dehyquart ® A-CA² | 2.0 |
| Salcare ® SC 96⁵ | 1.0 |
| Citric acid | 0.4 |
| Gluadin ® W40⁶ | 2.0 |
| Pyridoxine | 1.0 |
| Tartaric acid | 0.7 |
| Phenonip ®³ | 0.8 |
| Water | to 100 |

¹Cetylstearyl alcohol + 20 EO (INCI name: Ceteareth-20) (COGNIS)
²Trimethylhexadecylammonium chloride about 25% active substance (INCI name: Cetrimonium Chloride) (COGNIS)
³Hydroxybenzoic acid methyl ester-hydroxybenzoic acid ethyl ester-hydroxybenzoic acid propyl ester-hydroxybenzoic acid butyl ester-phenoxyethanol mixture (about 28% active substance; INCI name: Phenoxyethanol, Methylparaben, Ethylparaben, Propylparaben, Butylparaben) (NIPA)
⁴Mixture of alkyl polyglycoside and fatty acid monoglyceride (INCI name: Coco-Glucoside (and) Glyceryl Oleate)
⁵N,N,N-trimethyl-2[(methyl-1-oxo-2-propenyl)oxy]-ethanaminium chloride homopolymer (50% active substance; INCI name: Polyquaternium-37 (and) Propylene glycol Dicaprilate Dicaprate (and) PPG-1 Trideceth-6) (ALLIED COLLOIDS)
⁶Wheat protein hydrolyzate about 40% active substance (INCI name: Hydrolyzed Wheat Protein) (COGNIS)

2. Hair Rinse

| | |
|---|---|
| Eumulgin ® B2 | 0.3 |
| Cetyl/stearyl alcohol | 3.3 |
| Isopropyl myristate | 0.5 |
| Paraffin oil perliquidum 15 cSt. GP 9 | 0.3 |
| Sensomer ® 50⁷ | 2.5 |
| Dehyquart ® L 80⁸ | 0.4 |
| Lamesoft ® PO 65 | 1.5 |
| Cosmedia Guar ® C 261⁹ | 1.5 |
| Promois ® Milk-CAQ¹⁰ | 3.0 |
| Citric acid | 0.4 |
| Potassium Cocoyl Hydrolyzed Silk | 2.0 |
| Hexapeptide-2 | 0.3 |
| Poly-L-serine | 0.5 |
| Phenonip ® | 0.8 |
| Water | to 100 |

⁷Starch Hydroxypropyltrimonium Chloride, Ondeo Nalco, about 22% active substance, obtained from potato starch
⁸Bis(cocoylethyl)hydroxyethylmethylammonium metho-sulfate (about 76% active substance in propylene glycol; INCI name: Dicocoylethyl Hydroxyethylmonium Methosulfate, Propylene Glycol) (COGNIS)
⁹Guarhydroxypropyltrimethylammonium chloride; INCI name: Guar Hydroxypropyl Trimonium Chloride (COGNIS)
¹⁰INCI name: Cocodimonium Hydroxypropyl Hydrolyzed Casein (SEIWA KASEI)

3. Hair Tonic

| | |
|---|---|
| Dehyquart ® F75¹¹ | 4.0 |
| Cetyl/stearyl alcohol | 4.0 |
| Paraffin oil perliquidum 15 cSt GP 9 | 1.5 |
| Dehyquart ® A-CA | 4.0 |
| Cationic rice starch, MW 10000000, DS 0.25 | 7.5 |
| Lamesoft ® P065 | 1.0 |
| Salcare ® C 96 | 1.5 |
| Sericin | 0.1 |
| Promois ® Silk 1000 | 0.4 |
| D/L-Isoleucine | 2.5 |
| Glyoxylic acid | 0.5 |
| Amisafe-LMA-60 ®¹² | 1.0 |
| Gluadin ® W 20¹³ | 3.0 |
| Germall ® 115¹⁴ | 1.0 |
| Citric acid | 0.15 |
| Phenonip ® | 0.8 |
| Water | to 100 |

¹¹Fatty alcohols-methyltriethanolammonium methylsulfate dialkyl ester mixture (INCI name: Distearoylethyl Hydroxyethylmonium Methosulfate, Cetearyl Alcohol) (COGNIS)
¹²INCI name Hydroxypropyl Arginine Lauryl/Myristyl Ether HCl (Ajinomoto)
¹³Wheat protein hydrolyzate (20% active substance in water; INCI name: Aqua (and) Hydrolized Wheat Protein (and) Sodium Benzoate (and) Phenoxyethanol (and) Methylparaben (and) Propylparaben) (COGNIS)
¹⁴INCI name: Imidazolidinyl Urea (Sutton Laboratories)

4. Hair Tonic

| | |
|---|---|
| Dehyquart ® L80 | 2.0 |
| Cetyl/stearyl alcohol | 6.0 |
| Paraffin oil perliquidum 15 cSt GP 9 | 2.0 |
| Rewoquat ® W 75¹⁵ | 2.0 |
| Cosmedia Guar ® C261 | 0.5 |
| Lamesoft ® PO 65 | 0.5 |
| Sepigel ® 305¹⁶ | 3.5 |
| Honeyquat ® 50¹⁷ | 1.0 |
| Gluadin ® WQ | 2.5 |
| Gluadin ® W 20 | 3.0 |
| Cat. potato starch, MW 4000000, DS 0.5 | 3.5 |
| Hydrolyzed Sericin | 0.8 |
| L-Glycine | 0.4 |
| L-Alanyl-L-proline | 1.0 |
| L-Tyrosine | 0.2 |
| Citric acid | 0.15 |
| Phenonip ® | 0.8 |
| Water | to 100 |

¹⁵1-Methyl-2-nortallow alkyl-3-tallow fatty acid amidoethylimidazolinium methosulfate (about 75% active substance in propylene glycol; INCI name: Quarternium-27, Propylene Glycol) (WITCO)
¹⁶Copolymer of acrylamide and 2-acrylamido-2-methyl-propanesulfonic acid (INCI name: Polyacrylamide (and) C₁₃-C₁₄ Isoparaffin (and) Laureth-7) (SEPPIC)
¹⁷INCI name: Hydroxypropyltrimomum Honey (BROOKS)

5. Hair Tonic

| | |
|---|---|
| Dehyquart ® F75 | 0.3 |
| Salcare ® SC 96 | 5.0 |
| Gluadin ® WQ | 1.5 |
| Lamesoft ® PO 65 | 0.5 |
| Dow Corning ® 200 Fluid, 5 cSt.¹⁸ | 1.5 |
| Cat. cornstarch, MW 2000000, DS 0.75 | 2.0 |
| Gafquat ® 755N¹⁹ | 1.5 |
| Poly-D/L-alanine | 1.5 |
| Serine | 0.1 |
| Glycine methyl ester | 0.2 |
| Tyrosine methyl ester | 0.2 |
| Sericin | 0.2 |
| Biodocarb ®²⁰ | 0.02 |
| Perfume oil | 0.25 |
| Water | to 100 |

¹⁸Polydimethylsiloxane (INCI name: Dimethicone) (DOW CORNING)
¹⁹Dimethylaminoethyl methacrylate-vinylpyrrolidone copolymer, quaternized with diethyl sulfate (19% active substance in water; INCI name: Polyquaternium-11) (GAF)
²⁰3-Iodo-2-propynyl n-butylcarbamate (INCI name: Iodopropynyl Butylcarbamate) (MILKER & GRUNING)

6. Hair Tonic

| | |
|---|---|
| Sepigel ® 305 | 5.0 |
| Dow Corning ® Q2-5220²¹ | 1.5 |
| Promois ® MilkQ²² | 3.0 |
| Lamesoft ® PO 65 | 0.5 |
| Polymer P1 according to DE 3929173 | 0.6 |
| Genamin ® DSAC²³ | 0.3 |
| D/L-Methionine-S-methylsulfonium chloride | 1.8 |
| Hydrolyzed Sericin | 0.5 |
| Promois ® Silk 1000 | 1.0 |
| Cat. potato starch, MW 5000000, DS 2.0 | 4.5 |
| Phenonip ® | 0.8 |
| Perfume oil | 0.25 |
| Water | to 100 |

²¹Silicone-glycol copolymer (INCI name: Dimethicone Copolyol) (DOW CORNING)
²²INCI name Hydroxypropyltrimonium Hydrolyzed Casein about 30% active substance (SEIWA KASEI)
²³Dimethyldistearylammonium chloride (INCI name: Distearyldimonium Chloride) (CLARIANT)

7. Shampoo

| | |
|---|---|
| Texapon ® NSO²⁴ | 40.0 |
| Dehyton ® G²⁵ | 6.0 |
| Polymer JR 400 ®²⁶ | 0.5 |
| Cetiol ® HE²⁷ | 0.5 |
| Ajidew ® NL 50²⁸ | 1.0 |
| Lamesoft ® PO 65 | 3.0 |
| Sericin | 0.8 |
| Promois ® Silk 1000 | 2.0 |
| Gluadin ® WQT²⁹ | 2.5 |
| Gluadin ® W 20 | 0.5 |
| Panthenol (50%) | 0.3 |
| Cat. rice starch, MW 3000000, DS 0.5 | 5.0 |
| Casein | 2.0 |
| Vitamin E | 0.1 |
| Vitamin H | 0.1 |
| Glutamic acid | 0.2 |
| Citric acid | 0.5 |
| Sodium benzoate | 0.5 |
| Perfume | 0.4 |
| NaCl | 0.5 |
| Water | to 100 |

²⁴Sodium lauryl ether sulfate about 28% active substance (INCI name: Sodium Laureth Sulfate) (COGNIS)
²⁵INCI name: Sodium Cocoamphoacetate, about 30% active substance in water) (COGNIS)
²⁶Quaternized hydroxyethylcellulose (INCI name: Polyquaternium-10) (UNION CARBIDE)
²⁷Polyol fatty acid ester (INCI name: PEG-7 Glyceryl Cocoate) (COGNIS)
²⁸Sodium salt of 2-pyrrolidinone-5-carboxylic acid (50% active substance: INCI name: Sodium PCA) (AJINOMOTO)
²⁹INCI name: Hydroxypropyltrimonium Hydrolyzed Wheat Protein (COGNIS)

8. Shampoo

| | |
|---|---|
| Texapon ® NSO | 43.0 |
| Dehyton ® K³⁰ | 10.0 |
| Plantacare ® 1200 UP³¹ | 4.0 |
| Lamesoft ® PO 65 | 2.5 |
| Euperlan ® PK 3000³² | 1.6 |
| Arquad ® 316³³ | 0.8 |
| Polymer JR ® 400 | 0.3 |
| Gluadin ® WQ | 4.0 |
| Sensomer CI 50 | 2.5 |
| Lauryldimonium Hydroxypropyl Hydrolyzed Silk | 3.0 |
| Sodium Lauroyl Hydrolyzed Silk | 3.0 |
| Sericin | 10.0 |
| Lactic acid | 0.5 |
| Hydrolupin ® AA³⁴ | 0.5 |
| Malic acid | 0.5 |
| Glucamate ® DOE 120³⁵ | 0.5 |
| Sodium chloride | 0.2 |
| Water | to 100 |

³⁰INCI name: Cocamidopropyl Betaine about 30% active substance (COGNIS)
³¹C₁₂-C₁₆-fatty alcohol glycoside about 50% active substance (INCI name: Lauryl Glucoside) (COGNIS)
³²Liquid dispersion of pearl luster-imparting substances and amphosurfactant (about 62% active substance; CTFA name: Glycol Distearate (and) Glycerol (and) Laureth-4 (and) Cocoamidopropyl Betaine) (COGNIS)
³³Tri-C₁₆-alkylmethylammonium chloride (AKZO)
³⁴Amino acid mixture obtained by total hydrolysis of lupin protein, (INCI name Lupine Amino Acids) (CRODA)
³⁵Ethoxylated methylglucoside dioleate (CTFA name: PEG-120 Methyl Glucose Dioleate) (AMERCHOL)

9. Shampoo

| | |
|---|---|
| Texapon ® N 70³⁶ | 21.0 |
| Plantacare ® 1200 UP | 8.0 |
| Lamesoft ® PO 65 | 3.0 |
| Cat. potato starch, MW 3000000. DS 0.5 | 1.0 |
| Gluadin ® WQ | 1.5 |
| Promois ® Silk 1000 | 15.0 |
| Sericin | 10.0 |
| Cutina ® EGMS³⁷ | 0.6 |
| Honeyquat ® 50 | 2.0 |
| Ajidew ® NL 50 | 2.8 |
| Antil ® 141³⁸ | 1.3 |
| Crolastin ®³⁹ | 1.0 |
| Sodium chloride | 0.2 |
| Magnesium hydroxide | to pH 4.5 |
| Water | to 100 |

³⁶Sodium lauryl ether sulfate with 2 mol of EO about 70% active substance (INCI name: Sodium Laureth Sulfate) (COGNIS)
³⁷Ethylene glycol monostearate (about 25-35% monoester, 60-70% diester; INCI name: Glycol Stearate) (COGNIS)
³⁸Polyoxyethylene-propylene glycol dioleate (40% active substance; INCI name: Propylene Glycol (and) PEG-55 Propylene Glycol Oleate) (GOLDSCHMIDT)
³⁹Elastin hydrolyzate (INCI name: Hydrolyzed Elastin) (CRODA)

10. Shampoo

| | |
|---|---|
| Texapon ® K 14 S⁴⁰ | 50.0 |
| Dehyton ® K | 10.0 |
| Plantacare ® 818 UP⁴¹ | 4.5 |
| Lamesoft ® PO 65 | 2.0 |
| Potassium Cocoyl Hydrolyzed Silk | 5.0 |
| Palmitoyl Pentapeptide-2 | 2.5 |
| Polymer P1, according to DE 39 29 973 | 0.6 |
| Cutina ® AGS⁴² | 2.0 |
| D-Panthenol | 0.5 |
| Glucose | 1.0 |
| Sensomer CI 50 | 1.5 |
| Hydrosesame ® AA⁴³ | 0.8 |
| Salicylic acid | 0.4 |
| Sodium chloride | 0.5 |
| Gluadin ® WQ | 2.0 |
| Water | to 100 |

⁴⁰Sodium lauryl myristyl ether sulfate about 28% active substance (INCI name: Sodium Myreth Sulfate) (COGNIS)
⁴¹C₈-C₁₆-fatty alcohol glycoside about 50% active substance (INCI name: Coco Glucoside) (COGNIS)
⁴²Ethylene glycol stearate (about 5-15% monoester, 85-95% diester; INCI name: Glycol Distearate) (COGNIS)
⁴³INCI name: Water (and) Sesame Amino Acids (CRODA)

11. Hair Tonic

| | |
|---|---|
| Celquat ® L 200⁴⁴ | 0.6 |
| Luviskol ® K30⁴⁵ | 0.2 |
| D-Panthenol | 0.5 |
| Polymer P1, according to DE 39 29 973 | 0.6 |
| Dehyquart ® A-CA | 1.0 |
| Lamesoft ® PO 65 | 0.5 |
| Hydrosoy ® 2000⁴⁶ | 1.0 |
| Aspartic acid | 0.3 |
| Acetyl Hexapeptide-3 | 2.0 |
| Promois ® Silk 1000 | 5.0 |
| Gluadin ® W 40 | 1.0 |
| Cat. potato starch, MW 5000000. DS 0.25 | 2.5 |
| Natrosol ® 250 HR⁴⁷ | 1.1 |
| Gluadin ® WQ | 2.0 |
| Water | to 100 |

⁴⁴Quaternized cellulose derivative (95% active substance; CTFA name: Polyquaternium-4) (DELFT NATIONAL)
⁴⁵Polyvinylpyrrolidone (95% active substance; CTFA name: PVP) (BASF)
⁴⁶Protein hydrolyzate from soybeans (INCI name: Hydrolyzed Soy Protein) (CRODA)
⁴⁷Hydroxyethylcellulose (AQUALON)

12. Colour Cream

| | |
|---|---|
| C₁₂₋₁₈-fatty alcohol | 1.2 |
| Lanette ® O⁴⁸ | 4.0 |
| Eumulgin ® B 2 | 0.8 |
| Cutina ® KD 1649 | 2.0 |
| Lamesoft ® PO 65 | 4.0 |
| Sodium sulfite | 0.5 |
| L(+)-Ascorbic acid | 0.5 |
| Ammonium sulfate | 0.5 |
| 1,2-Propylene glycol | 1.2 |
| Polymer JR ® 400 | 0.3 |
| Cat. cornstarch, MW 2000000, DS 0.15 | 2.5 |
| p-Aminophenol | 0.35 |
| p-Toluylenediamine | 0.85 |
| 2-Methylresorcinol | 0.14 |
| 6-Methyl-m-aminophenol | 0.42 |
| Cetiol ® OE⁵⁰ | 0.5 |
| Honeyquat ® 50 | 1.0 |
| Ajidew ® NL 50 | 1.2 |
| Gluadin ® WQ | 1.0 |
| Crosilk Liquid ®⁵¹ | 0.5 |
| Promois ® Silk 1000 | 0.5 |
| Sericin | 0.3 |
| Ammonia | 1.5 |
| Water | to 100 |

⁴⁸Cetyl stearyl alcohol (INCI name: Cetearyl Alcohol) (COGNIS)
49Self-emulsifying mixture of mono-/diglycerides of higher saturated fatty acids with potassium stearate (INCI name: Glyceryl Stearate SE) (COGNIS)
⁵⁰Di-n-octyl ether (INCI name: Dicaprylyl Ether) (COGNIS)
⁵¹Mixture of amino acids obtained by total hydrolysis of silk protein (INCI name: Silk Amino Acids) (CRODA)

13. Developer Dispersion for Colour Cream 12.

| | |
|---|---|
| Texapon ® NSO | 2.1 |
| Hydrogen peroxide (50% strength) | 12.0 |
| Turpinal ® SL⁵² | 1.7 |
| Latekoll ® D⁵³ | 12.0 |
| Cat. tapioca starch, MW 2000000, DS 0.3 | 2.5 |
| Lamesoft ® PO 65 | 2.0 |
| Gluadin ® WQ | 0.3 |
| Salcare ® SC 96 | 1.0 |
| Aspartic acid | 0.1 |
| Sericin | 0.2 |
| Promois ® Silk 1000 | 0.4 |
| Crolastin ® | 0.8 |
| Water | to 100 |

⁵²1-Hydroxyethane-1,1-diphosphonic acid (60% active substance; INCI name: Etidronic Acid) (COGNIS)
⁵³Acrylic ester-methacrylic acid copolymer (25% active substance) (BASF)

14. Tinting Shampoo

| | |
|---|---|
| Texapon ® N 70 | 14.0 |
| Dehyton ® K | 10.0 |
| Akypo ® RLM 45 NV⁵⁴ | 14.7 |
| Plantacare ® 1200 UP | 4.0 |
| Lamesoft ® PO 65 | 3.0 |
| Polymer P1, according to DE 39 29 973 | 0.3 |
| Cat. potato starch, MW 3000000 | 2.5 |
| Cremophor ® RH 40⁵⁵ | 0.8 |
| Poly-L-serine | 0.3 |
| Hydrolyzed Sericin | 0.3 |
| Hydroxypropyltrimonium Hydrolyzed Silk | 3.0 |
| Benzoic acid | 0.3 |
| Poly-L-proline | 0.3 |
| Dye C.I. 12 719 | 0.02 |
| Dye C.I. 12 251 | 0.02 |
| Dye C.I. 12 250 | 0.04 |
| Dye C.I. 56 059 | 0.03 |
| Preservative | 0.25 |
| Perfume oil | q.s. |
| Eutanol ® G⁵⁶ | 0.3 |
| Gluadin ® WQ | 1.0 |
| Honeyquat ® 50 | 1.0 |
| Salcare ® SC 96 | 0.5 |
| Water | to 100 |

⁵⁴Lauryl alcohol + 4.5 ethylene oxide-acetic acid sodium salt (20.4% active substance) (CHEM-Y)
⁵⁵castor oil, hydrogenated + 45 ethylene oxide (INCI name: PEG-40 Hydrogenated Castor Oil) (BASF)
⁵⁶2-Octyldodecanol (Guerbet alcohol) (INCI name: Octyldodecanol) (COGNIS)

15. Cream Permanent Wave

Waving cream

| | |
|---|---|
| Plantacare ® 810 UP⁵⁷ | 5.0 |
| Thioglycolic acid | 8.0 |
| Turpinal ® SL | 0.5 |
| Ammonia (25% strength) | 7.3 |
| Ammonium carbonate | 3.0 |
| Cat. rice starch | 1.5 |
| Cetyl/stearyl alcohol | 5.0 |
| Lamesoft ® PO 65 | 0.5 |
| Guerbet alcohol | 4.0 |
| Salcare ® SC 96 | 3.0 |
| Gluadin ® WQ | 2.0 |
| Hydrolyzed Sericin | 0.3 |
| Hydroxypropyltrimonium Hydrolyzed Silk | 1.0 |
| Glutaric acid | 0.2 |
| Hydrotriticum ® 2000⁵⁸ | 0.5 |
| Perfume oil | q.s. |
| Water | to 100 |

⁵⁷C₈-C₁₀-Alkyl glucoside having a degree of oligomerization of 1.6 (about 60% active substance) (COGNIS)
⁵⁸Wheat protein hydrolyzate (INCI name: Hydrolyzed Wheat Protein) (CRODA)

Setting Solution

| | |
|---|---|
| Plantacare ® 810 UP | 5.0 |
| Hardened castor oil | 2.0 |
| Lamesoft ® PO 65 | 1.0 |
| Potassium bromate | 3.5 |
| Nitrilotriacetic acid | 0.3 |
| Citric acid | 0.2 |
| Merquat ® 550⁵⁹ | 0.5 |
| Hydagen ® HCMF⁶⁰ | 0.5 |
| Cat. cornstarch | 2.5 |
| Tartaric acid | 0.5 |
| Gluadin ® WQ | 0.5 |
| Cocodimonium Hydroxypropyl Silk Amino Acids | 0.3 |
| Hydrolyzed Sericin | 0.1 |
| D/L-Methionine S-methylsulfonium chloride | 0.3 |
| Perfume oil | q.s. |
| Water | to 100 |

⁵⁹Dimethyldiallylammonium chloride-acrylamide copolymer (8% active substance; INCI name: Polyquarternium 7) (MOBIL OIL)
⁶⁰Chitosan powder (INCI name: Chitosan) (COGNIS)

1. A cosmetic composition for improving the internal and/or external structure of keratinic fibers comprising a cationic starch derivative.

2. The composition of claim 1 wherein the cationic starch derivative is a polysaccaride obtained from potatoes, tapioca, cereals or corn.

3. The composition of claim 2 wherein the amount of cationic starch derivative present in the cosmetic composition is from about 0.05 to about 10.0%, by weight, based on the total weight of the composition.

4. The composition of claim 3 wherein the amount is from about 0.1 to about 5.0%, by weight.

5. The composition of claim 1 further comprising cationic, anionic, nonionic, zwitterionic and/or amphoteric surfactants.

6. The composition of claim 1 further comprising cationic, anionic, amphoteric and nonionic polymers.

7. A cosmetic composition for improving the fastness properties of dyed and/or bleached keratinic fibers comprising a cationic starch derivative.

8. The composition of claim 7 wherein the cationic starch derivative is a polysaccaride obtained from potatoes, tapioca, cereals or corn.

9. The composition of claim 8 wherein the amount of cationic starch derivative present in the cosmetic composition is from about 0.05 to about 10.0%, by weight, based on the total weight of the composition.

10. The composition of claim 9 wherein the amount is from about 0.1 to about 5.0%, by weight.

11. The composition of claim 7 further comprising cationic, anionic, nonionic, zwitterionic and/or amphoteric surfactants.

12. The composition of claim 7 further comprising cationic, anionic, amphoteric and nonionic polymers.

13. A composition for restructuring of keratinic fibers comprising a cationic starch derivative.

14. The composition of claim 13 wherein the cationic starch derivative is a polysaccaride obtained from potatoes, tapioca, cereals or corn.

15. The composition of claim 14 wherein the amount of cationic starch derivative present in the cosmetic composition is from about 0.05 to about 10.0%, by weight, based on the total weight of the composition.

16. The composition of claim 15 wherein the amount is from about 0.1 to about 5.0%, by weight.

17. The composition of claim 13 further comprising cationic, anionic, nonionic, zwitterionic and/or amphoteric surfactants.

18. The composition of claim 13 further comprising cationic, anionic, amphoteric and nonionic polymers.

19. A process for the treatment of skin or hair comprising adding to the hair or skin the composition of claim 1 and then rinsing the composition after about 1 to 45 minutes.
